(19) United States
(12) Patent Application Publication
(10) Pub. No.: US 2022/0152540 A1
(43) Pub. Date: May 19, 2022

(54) MICROWAVE ENHANCED AIR DISINFECTION SYSTEM

(71) Applicant: Vektra Systems LLC, Sacramento, CA (US)

(72) Inventors: Chang Yul Cha, Roseville, CA (US); Suk-Bae Cha, Tokyo (JP); George Crandell, Sacramento, CA (US); Craig Henricksen, Oakland, CA (US); William Walden, Fair Oaks, CA (US)

(21) Appl. No.: 17/522,691

(22) Filed: Nov. 9, 2021

Related U.S. Application Data
(60) Provisional application No. 63/113,687, filed on Nov. 13, 2020.
(60) Provisional application No. 63/166,004, filed on Mar. 25, 2021.

(51) Int. Cl. B01D 46/00; B01D 53/04; B01D 53/26; B01D 53/00; A61L 9/18

(52) U.S. Cl. CPC B01D 46/0028; A61L 2209/111; B01D 53/0438; B01D 53/0454; B01D 53/261; B01D 53/007; B01D 53/005; A61L 9/18; B01D 2253/108; B01D 2279/65; B01D 2279/35; B01D 2273/22; B01D 2253/25; B01D 2257/80; B01D 2259/40094; B01D 2259/806; B01D 2257/91; A61L 2209/14; B01D 46/0036

(57) ABSTRACT

A microwave enhanced air disinfection (MEAD) device includes a housing and a microwave generator coupled to the housing. The microwave generator is configured to generate microwave energy. The MEAD device further includes a multi-component filter disposed in the housing. The multi-component filter is configured to collect contaminants from airflow. At least a portion of the contaminants from the airflow is to be destroyed at least one of directly or indirectly via the microwave energy.

### RELATED APPLICATIONS

This application claims benefit of Provisional Application No. 63/113,687, filed Nov. 13, 2020, and Provisional Application No. 63/166,004, filed Mar. 25, 2021, the entire content of each is incorporated by reference herein.

### TECHNICAL FIELD

Embodiments of the present disclosure relate to an air disinfection systems, and in particular to microwave enhanced air disinfection systems.

### BACKGROUND

Air can include contaminants. Contaminants can include particulate matter, ground-level ozone, carbon, monoxide, sulfur dioxide, nitrogen dioxide, and lead. Other contaminants include microorganisms (e.g., living and non-living) and agents that cause infectious diseases.

### BRIEF DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIGS. 1A-B are block diagrams illustrating microwave enhanced air disinfection (MEAD) systems, according to certain embodiments.

FIGS. 2A-D illustrate multi-component filters of MEAD systems, according to certain embodiments.

FIGS. 3A-B are cross-sectional views of a MEAD system, according to certain embodiments.

FIGS. 4A-B are cross-sectional views of a MEAD system, according to certain embodiments.

FIGS. 5A-I illustrate MEAD systems, according to certain embodiments.

FIG. 6 is a block diagram illustrating a computer system, according to certain embodiments.

### DETAILED DESCRIPTION OF EMBODIMENTS

Embodiments described herein are related to microwave enhanced air disinfection (MEAD) systems.

Safe breathable air is a basic human need. The safety of indoor air is now one of the most important issues facing governments, business operators, and consumers worldwide. Even before the severe acute respiratory syndrome coronavirus 2 (SARS-CoV-2) (e.g., coronavirus disease 2019 (COVID-19), novel coronavirus) crisis began, indoor air quality was recognized as an emerging global health issue. The World Health Organization has estimated that one in every eight people die due to factors attributable to poor indoor air. However, since most of these deaths occur in developing countries, indoor air safety has not been a focus of global attention until the COVID-19 pandemic.

Air can include many contaminants including particulate matter (e.g., particles), ground-level ozone, carbon, monoxide, sulfur dioxide, nitrogen dioxide, lead, microorganisms (e.g., living and non-living organisms), viruses, allergens, and agents. Contaminants in the air can harm human health, harm the environment, and cause property damage.

Microorganisms (e.g., microscopic organisms) live in almost every habitat around the world. Pathogens (e.g., infectious agent, something that causes a disease, living and non-living organisms, etc.) include infectious microorganisms and agents, such as virus (e.g., non-enveloped virus, enveloped virus), bacterium, protozoan, prion, viroid, and fungus. For example, some pathogenic bacteria cause diseases such as plague, tuberculosis, and anthrax. In another example, some protozoan parasites cause diseases such as malaria, sleeping sickness, dysentery, and toxoplasmosis. In another example, some fungi cause diseases such as ring worm, candidiasis, or histoplasmosis. Some pathogenic viruses cause influenza virus (e.g., the flu), yellow fever, COVID-19, and the like.

COVID-19 and other diseases such as influenza and the common cold have been shown to be readily transmitted by airborne pathogens. Some pathogens are spread via small droplets produced by coughing, sneezing, and talking. The droplets travel through the air and some contaminate surfaces. People can become infected by coming into contact with the droplets in the air or by touching a contaminated surface and then touching their face (e.g., eyes, nose, and/or mouth). In some instances, pathogens may be spread by an infected person before and while showing symptoms.

Some pathogens (e.g., the influenza virus) spread around the world in periodical outbreaks, resulting in millions of cases of severe illness and hundreds of thousands of deaths. Some pathogens have vaccines or specific antiviral treatments, while others do not. Pandemics (e.g., COVID-19) are a spread by a pathogen causing a disease across a large region, affecting a substantial number of people within a short period of time.

Conventionally, air is periodically circulated through indoor areas (e.g., one or more rooms in a building). Conventional air circulation systems include a filter to trap some particles that are in the air that is being circulated. These conventional filters are periodically replaced. Conventional filters that do not cause much restriction on airflow trap less particles than conventional filters that cause more restriction on airflow. As filters trap more and more particles over time, the filters cause more and more restriction on air flow. Increased restriction on airflow can damage air treatment systems (e.g., cause freezing of cooling coils), decrease user comfort (e.g., provide less airflow), decrease air circulation, and the like. Conventional filters do not remove some contaminants from the air.

Conventional approaches are only partial solutions. Conventional filters capture but do not destroy contaminants (e.g., so that the contaminants no longer pose a threat) and require frequent replacement adding cost and creating a disposal hazard. Conventional filters are unable to capture small particles (e.g., smaller than 30 nm in size). Viruses like COVID-19 are small in size (e.g., significantly smaller than 30 nm) and are often found in droplets and particles also small in size (e.g., smaller than 30 nm in size) and can escape even the most robust conventional filtration systems. Further, as trapped moisture droplets dry and break-up, fragments can escape the filter and pose a significant additional infection risk. Some conventional filtration systems are fundamentally slow, often requiring hours to clean a room-size space after a single contamination. As a result, conventional approaches are unsuited for real-world applications. Because there is no effective means of neutralizing airborne COVID-19 available today, governments worldwide have been forced to implement policies to mitigate the spread of the disease, causing devastating economic damage and leaving businesses and consumers frantically searching for solutions. As such, there is an immediate and unmet need for air purifying products that can effectively destroy airborne contaminants like COVID-19.

The devices, systems, and methods disclosed herein provide a MEAD system. The MEAD system includes a housing (e.g., device housing, ducting), a microwave generator coupled to the housing, and a multi-component filter disposed in the housing. In some embodiments, the multi-component filter includes discrete layers (e.g., a metal oxide layer, a molecular sieve layer, and/or a high-efficiency particulate air (HEPA) filter). In some embodiments, the multi-component filter includes a heterogeneous mix of two or more filter materials that each perform a different function (e.g., metal oxide configured to remove living and non-living microorganisms from the airflow mixed with zeolites configured to remove volatile organic compounds (VOCs) from the airflow, etc.).

The microwave generator generates microwave energy. The multi-component filter collects contaminants from airflow. At least a portion of the contaminants are destroyed at least one of directly or indirectly via the microwave energy. In some embodiments, at least a portion of the multi-component filter is heated by the microwave energy to destroy (e.g., oxidize, destroy, destroy cell structure of) contaminants from the airflow (e.g., contaminants directly destroyed via microwave energy). In some embodiments, at least a portion of the multi-component filter (e.g., zeolites, metal oxides) is activated via the multi-component filter to destroy contaminants (e.g., destroy microbes, oxidize VOCs, etc.) from the airflow (e.g., contaminants indirectly destroyed via microwave energy). In some embodiments, one or more properties of the multi-component filter (e.g., zeolites, metal oxides) may remove (e.g., destroy) contaminants (e.g., with or without airflow). In some embodiments, the microwave energy catalyzes reactions (e.g., with temperatures lower than conventional temperatures used to produce reactions, provides lower temperature of reaction, directly and/or indirectly destroys contaminants). In some embodiments, the contaminants are destroyed by one or more reactions (e.g., substantially simultaneous reactions, destroying via heating and activated portions of the multi-component filter). The contaminants on the heated portion of the multi-component filter are destroyed and off-gassed.

The housing receives airflow (e.g., via a fan coupled to the housing). The airflow cools the microwave generator and the multi-component filter removes contaminants from the airflow. In some embodiments, a first fan (e.g., ventilation fan) is used to provide airflow through the housing and a second fan (e.g., cooling fan) is configured to cool the microwave generator (e.g., magnetron). In some embodiments, the first fan (e.g., ventilation fan) is turned off during heating of the multi-component filter. The second fan (e.g., cooling fan) may be disposed in a microwave housing (e.g., that houses the microwave generator).

The systems, devices, and methods disclosed herein have advantages over conventional solutions. The MEAD system removes more contaminants, removes smaller contaminants, and destroys contaminants compared to conventional systems that trap less contaminants, do not trap as small of contaminants, and do not destroy the contaminants. This allows the MEAD system to improve human health, improve the indoor environment, and cause less property damage compared to conventional system. The MEAD system destroys contaminants by heating the multi-component filter via microwave energy, by activating one or more portions (e.g., metal oxide, zeolites, etc.) of the multi-component filter via microwave energy, and so forth. The technology has been shown to kill aerosolized biological agents like Escherichia coli (E. coli), Escherichia virus MS2, and Bacillus Subtilis, which are commonly used to model COVID-19 and other dangerous pathogens, in 90 seconds, which is much faster (e.g., 20-50 times faster) than conventional systems. This allows the MEAD system to provide real-time purification of indoor air. Destruction of contaminants by the MEAD system avoids frequent filter replacement of conventional systems and avoids air restriction caused by filters that need to be replaced in conventional systems. This also allows the MEAD system to have thinner filters than filters in some conventional systems, which allows the MEAD system to have less restriction on airflow. The reduced restriction on airflow of the MEAD system decreases damage to air treatment systems, increases air circulation, and increases user comfort. The MEAD system may generate microwave energy intermittently via the microwave generator which decreases energy consumption.

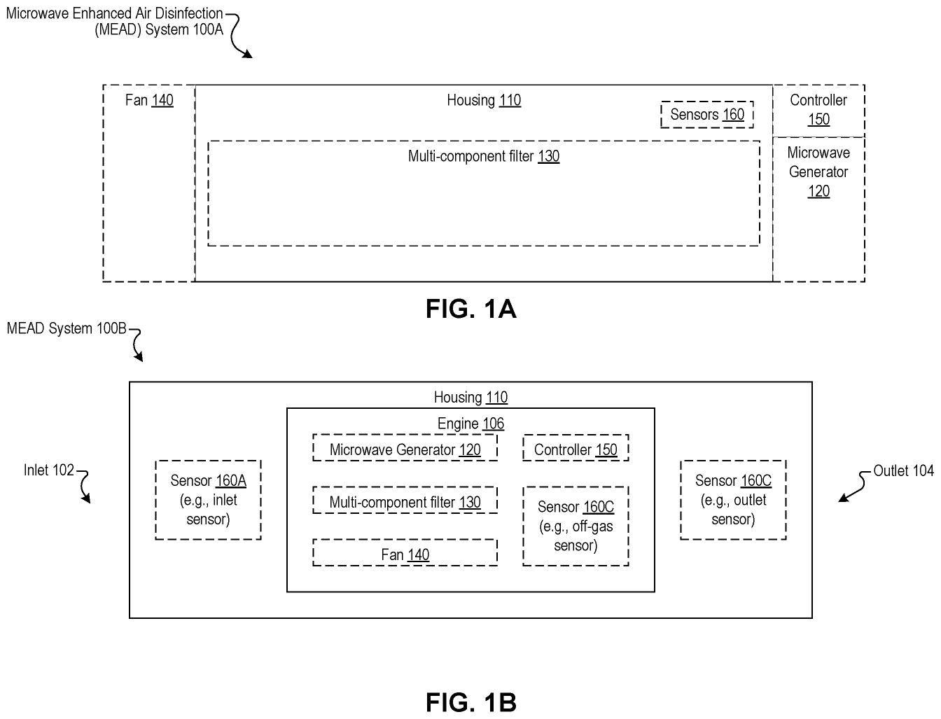

FIGS. 1A-B are block diagrams illustrating a MEAD systems 100A-B (hereinafter MEAD system 100) (e.g., a MEAD device), according to certain embodiments.

The MEAD system 100 includes a housing 110. In some embodiments, the MEAD system 100 is a device and the housing 110 is the device housing, where components of the MEAD system 100 are included in the housing 110 and/or are attached to the housing 110. In some embodiments, the housing 110 is or includes ducting of a ventilation system and components of the MEAD system 100 are included in the housing 110 and/or are attached to the housing 110. In some embodiments, the MEAD system 100 has one or more components that are coupled (e.g., electrically coupled, fluidly coupled, etc.) to each other without being attached to the housing 110 and/or disposed in the housing 110.

The MEAD system 100 includes at least one microwave generator 120 (e.g., microwave generator with magnetron tube, solid state microwave generator, solid state digital power supply, etc.) that is coupled to the housing 110. In some embodiments, the microwave generator 120 is disposed in the housing 110. In some embodiments, the microwave generator 120 is attached to the housing 110. The microwave generator 120 generates microwave energy that is transmitted into the housing 110. In some embodiments, the MEAD system 100 includes a microwave reflective enclosure (e.g., the housing 110 is a microwave reflective enclosure, a microwave reflective enclosure is disposed in the housing, etc.). In some embodiments, the microwave reflective enclosure includes an inlet microwave screen and an outlet microwave screen. In some embodiments, the inlet microwave screen and/or the outlet microwave screen are part of the multi-component filter 130. The microwave reflective enclosure prevents microwave energy from exiting the MEAD system 100. In some embodiments, the microwave generator 120 generates microwave energy intermittently (e.g., based on a schedule, based on sensor data, based on instructions, intermittent microwave energy operation, etc.). In some embodiments, the microwave generator 120 generates microwave energy continuously (e.g., continuous operation).

The MEAD system 100 includes a multi-component filter 130 that is disposed in the housing 110 (or at least partially disposed in the housing 110). Airflow passes through the multi-component filter 130 and contaminants from the airflow are trapped by the multi-component filter 130. At least a portion of the multi-component filter 130 is configured to be heated and/or activated by the microwave energy generated by the microwave generator 120 to remove (e.g., oxidize, destroy, off-gas, etc.) contaminants from the airflow (e.g., contaminants trapped in the multi-component filter 130). The contaminants are heated, destroyed, and/or off-gassed.

In some embodiments, the multi-component filter 130 is made of one or more filter materials (e.g., filter matrix). In some embodiments, the multi-component filter 130 includes a desiccant material (e.g., desiccating material, hydrophilic desiccating material) configured to absorb moisture including contaminants. The desiccant material removes moisture droplets (e.g., aerosols, water vapor, etc.) from the airflow. The moisture droplets may carry pathogens (e.g., virus, live virus). In some embodiments, water vapor contains most of the virus (e.g., live virus). The desiccant material can include silica gel, a polyacrylate, sodium polyacrylates, super-absorbent polymer (SAP), anionic polyelectrolyte, potassium SAP, lithium SAP, ammonium SAP, super-absorbent nanofiber (SAN), poly(vinyl alcohol) (PVA) (polymer matrix), SAP combined with PVA, hydrogel, clay-polymer hydrogel, clay, polyethylene oxide (PEO), sodium polyacrylates (PAAS), metal ions, chitosan, chitosan/sodium polyacrylates polyelectrolyte complex hydrogels (CPG), epichlorohydrin (ECH), activated charcoal, calcium sulfate, calcium chloride, molecular sieve (e.g., zeolite), a desiccant coating (e.g., on fiber, on a fibrous filter, on a HEPA filter, etc.), powder, zeolite, and/or other desiccant or hydrophilic materials. In some embodiments, the desiccant material is a coating on a material. For example, the desiccant material can be sprayed as resin on fiber. In some embodiments, the desiccant material is a coating for a fibrous filter (e.g., a high efficiency particulate air (HEPA) filter, fibrous filter with coating of desiccant material, HEPA filter with a coating of desiccant material, etc.). In some embodiments, the desiccant material is disposed in an enclosure (e.g., perforated enclosure, bag, enclosure similar to a flour bag, etc.). In some embodiments, the desiccant material has antimicrobial features. In some embodiments, the desiccant material collects contaminants and the contaminants are destroyed via one or more of heat, microwave energy, and/or material properties of the desiccant material.

Conventionally, a desiccant material may quickly saturate and lose efficacy. The MEAD system 100 uses microwave energy to periodically dry the materials (e.g., desiccant material, multi-component filter 130, etc.) and regenerate the materials. The microwave energy regenerates the desiccant material by causing the moisture to become steam to exit the MEAD system 100. The microwave energy causes the moisture to become steam and destroys contaminants from the moisture without directly heating the desiccant material.

In some embodiments, the desiccant material includes spherical beads (e.g., of silica gel, a polyacrylate, etc.) that are about 1-8 millimeters (mm) in diameter (e.g., 2-5 mm, 3-5 mm, or 4-8 mm in diameter). In some embodiments, the desiccant material include powder (e.g., about 100 to 500 microns in diameter). In some embodiments, the desiccant material includes different sizes of material (e.g., two or more of powder, beads, pellets, etc.). In some embodiments, the desiccant material is formed into shapes (e.g., capsules, pellets, etc.) that are adhered to each other (e.g., glued together) or placed in a semipermeable membrane. In some embodiments, the desiccant material is placed in a structure (e.g., honeycomb structure). The structure may be made of ceramic, aluminum mesh, etc. The structure may be coated. In some examples, a structure forms cavities (e.g., hexagon-shaped, pentagon-shaped, rectangular-shaped, etc.) and the desiccant material (e.g., in the form of pills, capsules, pellets, beads, powder, etc.) is placed in the cavities of the structure. The structure may conduct heat through the desiccant material evenly.

In some embodiments, the desiccant material (e.g., silica gel, a polyacrylate, beads, powder) of the multi-component filter 130 does not absorb microwave energy. The moisture collected in the desiccant material absorbs the microwave energy. The microwave energy may cause the moisture to become steam (e.g., vaporize the moisture) and destroy contaminants (e.g., micro bodies, viruses, pathogens, etc.) in the moisture without affecting the effectiveness (e.g., moisture absorption properties) of the desiccant material (e.g., silica gel, a polyacrylate, etc.). In some embodiments, both the desiccant material and the moisture collected in the desiccant material absorb microwave energy (e.g., both are heated by the microwave energy).

In some embodiments, the multi-component filter 130 includes a first silicon carbide (SiC) layer configured to absorb the microwave energy to destroy first contaminants, a zeolites and metal oxides layer configured to catalyze a reaction to destroy second contaminants, a desiccant material layer (e.g., silica gel, a polyacrylate, SAP, etc.) configured to absorb moisture including third contaminants (e.g., the third contaminants are to be destroyed responsive to the microwave energy causing the moisture to become steam), and/or a second SiC layer configured to absorb the microwave energy to destroy fourth contaminants. In some embodiments, the zeolites and metal oxides layer and the desiccant material layer are disposed between (e.g., sandwiched between) the first SiC layer and the second SiC layer (e.g., heat receptive material). In some embodiments, there are discrete layers, mixed layers, or a mixture of discrete and mixed layers. In some embodiments, a fibrous filter (e.g., HEPA filter, fibrous filter with coating of desiccant material, HEPA filter with a coating of desiccant material, etc.) is disposed as the last layer of the multi-component filter 130 that airflow goes through before exiting the housing 110. In some embodiments, airflow going through the multi-component filter 130 first goes through a first silicon carbide layer, then a zeolite layer, then a desiccant material (e.g., silica gel, a polyacrylate, etc.) layer, then a second silicon carbide layer, and then a HEPA filter. In some embodiments, producing of microwave energy by the microwave generator 120 while providing airflow via the fan 140 provides multiple opportunities to destroy contaminants. The first silicon carbide layer is heated by the microwave energy and may destroy a contaminant, the moisture in the desiccant (e.g., silica gel, a polyacrylate, etc.) layer is heated (e.g., to a greater temperature than the temperature of the first silicon carbide layer) and may destroy the contaminant, and the second silicon carbide layer is heated by the microwave energy and may destroy the contaminant. As the contaminant flows through the different layers, the contaminant may be destroyed by any of the layers (e.g., if the contaminant is not destroyed with one of the first layers, the contaminant can be destroyed by one of the later layers).

In some embodiments, one or more materials (e.g., zeolites and/or other materials) in the multi-component filter 130 are coated with metal oxides to catalyze reactions in the MEAD system 100 under microwave energy. The zeolites coated with metal oxides may provide a catalytic affect.

In some embodiments, the multi-component filter 130 includes a zeolite layer to collect VOCs, a silicon carbide heating foam matrix layer, and/or a desiccant material layer. The MEAD system 100 may capture moisture via the multi-component filter 130 (e.g., desiccant material) over a period of time (e.g., a predetermined number of hours), generate the microwave energy which heats the collected moisture (e.g., heat the collected moisture to 300° F. which kills micro bodies, pathogens, viruses, etc.) to produce steam, take a reading of amount of humidity (e.g., produced by the steam), and release the humidity out of the MEAD system. When the humidity is released, the humidity may contain the destroyed contaminants (e.g., dead micro bodies). The zeolites of the multi-component filter 130 may collect the VOCs, break the VOCs up into smaller VOCs, and oxidize the broken down smaller VOCs. In some embodiments, the airflow through the MEAD system 100 carries the heated moisture (e.g., steam, humidity, dead micro bodies, etc.) out of the MEAD system 100 (e.g., from the desiccant material).

In some embodiments, the multi-component filter 130 includes one or more materials that can capture contaminants (e.g., moisture droplets, dust, etc.) and one or more of the materials can absorb heat to destroy one or more of the contaminants. In some embodiments, the multi-component filter 130 is a multi-ply filter. The multi-component filter 130 may include a pre-filter (e.g., first layer, coating, silicon carbide, silicon carbide coating, etc.) that can capture contaminants and can be heated by microwave energy to destroy contaminants. The pre-filter may be coupled (e.g., glued, adhered, a coating sprayed onto, etc.) a backing layer. The backing layer may be a high-temperature capacity filter (e.g., a filter that can be reused after heating). The pre-filter may not capture fine dust and may provide a MERV 6-8 filter rating performance. The backing layer may provide MERV 10-12 performance to capture fine dust. The multi-component filter 130 including a pre-filter coupled to the backing layer may meet a at least a MERV 13 performance. For different applications (e.g., different MERV rating requirements, different pressure drop capacities of ventilation systems 501), different backing layers may be used. The pre-filter may remain the same for different applications.

The backing layer may be stable at high temperatures (e.g., can continue to be used subsequent to being exposed to high temperatures, maintain same MERV rating subsequent to being exposed to high temperatures, maintain the same structural and functional properties subsequent to be exposed to high temperatures). In some embodiments, the backing layer is stable up to at least 80 degrees Celsius. In some embodiments, the backing layer is stable up to at least 90 degrees Celsius. In some embodiments, the backing layer is stable up to at least 100 degrees Celsius. In some embodiments, the backing layer is stable at above 100 degrees Celsius. In some embodiments, the backing layer is stable at above 150 degrees Celsius. In some embodiments, the backing layer is stable responsive to repeatedly being exposed to microwave energy.

The multi-component filter 130 may be a multi-ply (e.g., 2-ply) filter that includes a pre-filter (e.g., silicon carbide coating, active layer) configured to absorb microwave energy and one or more backing layers. The pre-filter (e.g., coating) may perform moisture gathering and destruction of micro-organisms.

In some embodiments, the pre-filter (e.g., coating) is used with a first backing layer to provide at least a MERV 8 performance. In some embodiments, the pre-filter is used with a second backing layer to provide at least a MERV 13 performance. The backing layer may filter particulates (e.g., dust).

In some embodiments, the pre-filter is a coating that is applied to the backing layer. In some embodiments, the pre-filter is an open material that is spongy (e.g., about 1 inch thick that has silicon carbides and/or other materials sprayed on the open material). The pre-filter can have up to MERV 8 filter rating performance. To achieve MERV 13, a backing layer may be used in conjunction with the pre-filter. In some embodiments, the pre-filter is folded to form a pleated filter that supports MERV 13 cloth material (e.g., MERV 13 backing layer).

In some embodiments, the multi-component filter 130 includes a metal screen (e.g., chicken wire) to keep pressure drop from collapsing the multi-component filter 130. The pre-filter may include a metal screen that has a coating (e.g., silicon carbide).

In some embodiments, the multi-component filter 130 provides from MERV 8 to HEPA filter rating and has a high-temperature capability of up to about 100 degrees Celsius.

In some embodiments, The MEAD system 100 includes inlet and outlet microwave screens (e.g., grating to block microwave leakage) that are used in conjunction with the multi-component filter 130. An inlet microwave screen can be disposed proximate an inlet side of the multi-component filter 130 and an outlet screen can be disposed proximate an outlet side of the multi-component filter. Each microwave screen may be a protective mesh screen forming holes. The size and spacing of the holes in the microwave screen may reflect microwave energy to maintain the microwave energy within the multi-component filter 130 (e.g., to prevent the microwaves from leaking) while allowing airflow through the multi-component filter 130. In some embodiments, the inlet microwave screen is integrated into the multi-component filter 130. In some embodiments, the inlet microwave screen is coated with a microwave-absorbing material (e.g., silicon carbide) so that the inlet microwave screen heats responsive to receiving microwave energy to destroy contaminants. In some embodiments, the multi-component filter 130 includes a pre-filter that includes the inlet microwave screen coated with a microwave-absorbing material (e.g., silicon carbide), a backing layer coupled to the pre-filter, and an outlet microwave screen, where the backing layer is disposed between the pre-filter and the outlet microwave screen.

In some embodiments, the MEAD system 100 includes sensors 160 configured to provide sensor data (e.g., humidity data, resistance data, voltage data, imaging data, weight data, etc.) and a controller 150 that determines, based on the sensor data, that the desiccant material is to be regenerated and causes the microwave generator 120 to generate the microwave energy to regenerate the desiccant material. In some embodiments, the electrical resistance, voltage, color, humidity, weight, etc. of the desiccant material changes as the desiccant material goes from a substantially dry state to a substantially saturated state.

In some embodiments, the controller 150 receives, from one or more sensors 160 (e.g., at the inlet to the MEAD system 100, at the outlet to the MEAD system 100, within the MEAD system 100, etc.), sensor data indicative of humidity and/or temperature: of airflow into the MEAD system 100; inside the MEAD system 100 (e.g., with and/or without microwave energy being generated); and/or of airflow out of the MEAD system 100 (e.g., with and/or without microwave energy being generated). In some embodiments, the controller 150 determines, based on the sensor data, how much moisture the MEAD system 100 is retaining based on a difference between humidity in compared to humidity out. Responsive to the amount of moisture retained by the MEAD system 100 meeting a threshold value, the controller 150 may cause the microwave generator 120 to generate microwave energy.

In some embodiments, the desiccant material is periodically (e.g., a few minutes every hour, based on sensor data, etc.) regenerated (e.g., by microwave energy, by heat, etc.) to dehydrate the desiccant material (e.g., restore to normal, dehydrate aerosol and/or moisture in air, disintegrate micro bodies).

In some embodiments, the multi-component filter 130 is made of two or more filter materials, where each of the filter materials has a different function. In some embodiments, the multi-component filter 130 has two or more layers, where each of the layers is made of a different filter material. In some embodiments, the multi-component filter 130 uses one or more heterogeneous structures instead of or in addition to discrete filter layers. In some embodiments, the multi-component filter 130 is a heterogeneous mix (e.g., heterogeneous structure) of two or more filter materials that each have a different function. In some embodiments, the multi-component filter 130 includes one or more of a pre-filter, a microwave-absorbing material, a metal oxide (e.g., copper oxide, zinc oxide, titanium oxide, etc.), a metal carbide (e.g., silicon carbide, etc.), zeolites, a molecular sieve, a material without organic binders, a material with inorganic binders, a HEPA filter, and/or the like. In some embodiments, a layer of metal oxide is located closest to the microwave energy (e.g., is heated and/or activated the most), a HEPA filter layer is located furthest from the microwave energy (e.g., heated and/or activated the least, not heated and/or activated), and a zeolite layer is located between the metal oxide layer and the HEPA filter layer. In some embodiments, the metal layer is used to remove and destroy living and non-living microorganisms, the molecular sieve (e.g., zeolite layer) is used to remove VOCs, and the HEPA filter layer is used to remove remaining contaminants.

In some embodiments, the multi-component filter 130 is less than about 4 inches deep (e.g., less than 4 inches from where airflow enters the multi-component filter to where the airflow leaves the multi-component filter to exit the MEAD system 100). In some embodiments, the multi-component filter is less than about 3 inches deep. In some embodiments, the multi-component filter is less than about 2 inches deep. In some embodiments, the multi-component filter is about 2 to 4 inches deep. In some embodiments, the multi-component filter is 12 to 16 inches in length (e.g., the waveguide is 12 to 16 inches in length).

In some embodiments, a fan 140 provides airflow through the MEAD system 100 (e.g., the MEAD system 100 has a fan 140 coupled to the housing 110). In some embodiments, the MEAD system 100 has a fan 140 disposed within the housing 110. In some embodiments, the fan 140 provides the airflow into the housing 110 to be filtered by the multi-component filter 130 and the same fan 140 provides the airflow to cool the microwave generator 120. In some embodiments, fan 140 (e.g., ventilation fan that turns off during heating of the multi-component filter 130) provides airflow into the housing 110 and a second fan (e.g., cooling fan disposed in housing of the microwave generator 120) provides airflow to cool the microwave generator 120 (e.g., magnetron). In some embodiments, the MEAD system 100 does not have a fan (e.g., airflow is provided by a component outside of the MEAD system 100, such as by a blower of a heating ventilation and air conditioning (HVAC) system) to provide airflow through the housing 110 (e.g., MEAD system 100 may have a fan in the housing of the microwave generator 120 to cool the microwave generator 120). In some embodiments, the fan 140 (e.g., a suction fan) pulls the airflow into the housing 110 and causes the airflow to exit the housing 110 through the fan 140 (e.g., airflow goes through multi-component filter 130 before going through fan 140). In some embodiments, the fan 140 pushes the airflow into the MEAD system 100 and causes the airflow to exit the MEAD system 100 through the housing 110 (e.g., airflow goes through fan 140 before going through multi-component filter 130). In some embodiments, the fan 140 is configured to switch operation between pushing airflow and pulling airflow (e.g., to loosen contaminants in the multi-component filter 130). In some embodiments, the MEAD system 100 includes a pressure sensor to measure pressure drop across the multi-component filter 130. Responsive to the controller 150 determining, based on pressure data from the pressure sensor, that the pressure drop meets a threshold pressure drop, the controller 150 may cause one or more corrective actions (e.g., cause the fan 140 to increase airflow, cause the fan 140 to alternate airflow between pushing and pulling, provide an alert to clean or replace a portion of the MEAD system 100, etc.).

In some embodiments, the fan 140 is a quiet fan to pull air through the MEAD system 100. In some embodiments, the multi-component filter 130 includes a HEPA filter that removes about 99.97% of all small particles before discharge. In some embodiments, the multi-component filter 130 includes a filter matrix that effectively collects aerosols, odors, and other violates. In some embodiments, the filter is combined with materials (e.g., via inorganic binders) that react to microwave energy and are activated (e.g., heat to temperatures high enough) to destroy contaminants, such as viruses and VOCs. The microwave generator 120 (e.g., with a waveguide and/or magnetron tube) is used to distribute microwave energy evenly across filter materials of the multi-component filter 130. In some embodiments, contaminants (e.g., virus aerosols and VOCs) are collected on the multi-component filter 130 (e.g., filtration media) that can be heated and/or activated by microwave energy (e.g., microwaves) on a periodic cycle so that the microwave system is not operating continuously. In some embodiments, the MEAD system 100 operates an alternating adsorption-microwave regeneration cycle (e.g., multi-component filter 130 adsorbs contaminants and then the microwave generator 120 generates microwave energy to destroy the contaminants on the multi-component filter 130 to regenerate the multi-component filter 130).

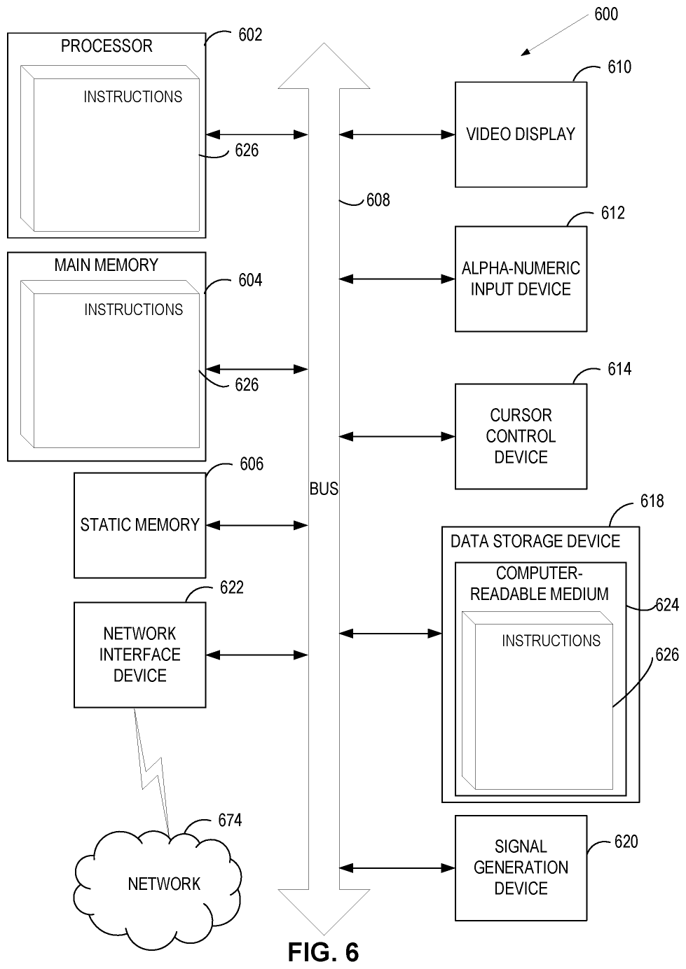

In some embodiments, the MEAD system 100 includes a controller 150 disposed in the housing 110 or coupled to the housing 110. In some embodiments, the microwave generator 120 includes a controller 150. The controller 150 includes one or more of a processing device, memory, sensors, wireless component, a user interface, and/or the like. In some embodiments, the controller 150 includes one or more of the components of computer system 600 of FIG. 6. In some embodiments, the controller actuates (e.g., turns on, turns off, adjusts fan speed, adjusts microwave energy generation, etc.) the microwave generator 120 and/or fan 140 based on one or more of a schedule, user input, sensor data, etc.

In some embodiments, the MEAD system 100 includes one or more sensors 160 coupled to or within the housing 110. In some embodiments, the one or more sensors 160 are disposed in the airflow after one or more portions of the multi-component filter 130 (e.g., after the airflow has been at least partially filtered). As the contaminants are trapped in the multi-component filter 130 and destroyed by the microwave energy heating and/or activating the multi-component filter 130, the contaminants are off-gassed. In some embodiments, the one or more sensors 160 are located to provide sensor data associated with the off-gassed contaminants.

In some embodiments, the fan 140 is disposed at a first distal end of the housing 110 and the microwave generator 120 is disposed at a second distal end of the housing 110 (e.g., see FIG. 1A). The fan 140 may pull airflow into the MEAD system via the housing 110 (e.g., the airflow exits through the fan 140) and/or the fan 140 may provide airflow into the MEAD system through the fan 140 (e.g., the airflow exits through the housing 110).

In some embodiments, the MEAD system 100 includes an inlet 102 (e.g., large airflow inlet) and an outlet 104 (e.g., large airflow outlet) that are substantially in line with each other (e.g., the inlet and the outlet are disposed along a common axis, a central axis substantially runs through a center of the inlet and a center of the outlet, see FIG. 1B, etc.). One or more components (e.g., an engine 106) may be disposed between the inlet 102 and the outlet 104 (e.g., between the inlet and outlet that are in line with each other). The engine 106 may include one or more of the microwave generator 120, multi-component filter 130, fan 140, controller 150, one or more sensors 160, etc.

In some embodiments, the sensors 160 include a sensor 160A is disposed proximate an inlet (e.g., inlet 102, housing 110) of the MEAD system 100, a sensor 160B is disposed proximate the off-gassing (e.g., multi-component filter 130, a portion of the multi-component filter 130 that reaches a higher temperature than other portions of the multi-component filter 130 to trigger combustion, etc.), and a sensor 160C located proximate the outlet (e.g., outlet 104, fan 140) of the MEAD system 100. The controller 150 may receive sensor data from the sensors 160 and cause a corrective action based on the sensor data or differences between the sensor data from different sensors 160. In some examples, responsive to determining, based on sensor data (e.g., off-gassing sensor data) from sensor 160B, that a threshold amount of contaminants or a certain type of contaminants are in the airflow, the controller 150 may cause the MEAD system 100 to continue operating (e.g., generating microwave energy and airflow, increase power provided to the microwave generator 120, increase airflow, etc.). Responsive to determining, based on sensor data from sensor 160B, that a threshold amount of contaminants or certain types of contaminants are not in the airflow, the controller 150 may cause the MEAD system 100 to stop or slow down operation (e.g., decrease power to microwave generator 120, decrease airflow via fan 140, stop generation of microwave energy and/or airflow, etc.).

In some examples, responsive to determining, based on sensor data (e.g., inlet sensor data) from sensor 160A and sensor data (e.g., outlet sensor data 160C) from sensor 160C, a difference value that exceeds a threshold difference value, the controller may cause the MEAD system 100 to continue operating (e.g., generating microwave energy and airflow). Responsive to determining, based on sensor data from sensors 160A and 160C, that a threshold difference value is not met, the controller 150 may cause the MEAD system 100 to stop or slow down operation (e.g., decrease power to microwave generator 120, decrease airflow via fan 140, stop generation of microwave energy and/or airflow, etc.).

In some embodiments, the controller 150 may cause the fan 140 to reverse airflow (e.g., inlet 102 is used as an outlet and outlet 104 is used as an inlet). Responsive to reversing airflow, the controller 150 may use sensor data from sensor 160C as inlet sensor data and may use sensor data from sensor 160A as outlet sensor data.

In some embodiments, the controller 150 may cause the MEAD system 100 to operate continuously (e.g., generate microwave energy via microwave generator 120 and generate airflow via fan 140 responsive to being turned on). In some embodiments, the controller 150 may cause the MEAD system 100 to operate intermittently (e.g., based on a timer, based on a schedule, based on sensor data, etc.).

In some embodiments, one or more MEAD systems 100 communicate, via a network, with a processing device (e.g., a server device, another MEAD system 100, client device, gateway device, etc.) that is remote from the one or more MEAD systems 100. The processing device may receive sensor data from the one or more MEAD systems 100 and provide instructions to (e.g., control, direct operation of) one or more MEAD systems 100. In some examples, responsive to receiving sensor data indicative of a certain contaminant (e.g., influenza, etc.), the processing device may cause multiple MEAD systems 100 (e.g., in a region, in a space, in a building) to perform an operation (e.g., increased power to the microwave generator 120, increased airflow, more frequent operation, etc.). In some examples, the processing device controls MEAD systems 100 located in a common space based on sensor data. The processing device may cause one MEAD system 100 to have a first operation (e.g., higher airflow, higher power to microwave generator 120) and cause other MEAD systems 100 in the same space to have a second operation (e.g., not operating, lower airflow, lower power to microwave generator 120) so that contaminants are destroyed without overworking all of the MEAD systems 100. The processing device may alternate which MEAD system 100 has the first operation to lessen wear-and-tear on a single MEAD system 100.

In some embodiments, the MEAD system 100 uses one or more products (e.g., multi-component filter 130, microwave generator 120, etc.) and/or one or more processes (e.g., using microwave energy generated by the microwave generator 120 to destroy contaminants trapped in the multi-component filter 130, controller 150 using sensor data from sensors 160 to control fan 140 and/or microwave generator 120 to destroy contaminants) relating to COVID-19 (e.g., destroying COVID-19 from the airflow) that is subject to an applicable Food and Drug Administration (FDA) and/or Environmental Protection Agency (EPA) approval for COVID-19 use.

In some embodiments, the microwave generator 120 provides microwave energy (e.g., radiofrequency microwave energy) through one or more waveguides (e.g., slot waveguide antennas) to the multi-component filter 130 to purify an airflow (e.g., air stream) containing contaminants (e.g., hazardous materials, organic vapors, etc.) and the multi-component filter 130 is regenerated without physical removal from the MEAD system 100.

The multi-component filter 130 may adsorb contaminants (e.g., organics) from contaminated airflow that passes through the multi-component filter 130 to purify the airflow. Saturation of the multi-component filter 130 (e.g., with contaminants) may eventually occur. Conventionally, a filter is replaced or the filter is removed for desorption via steam. The MEAD system 100 performs desorption of the multi-component filter 130 in situ by providing microwave energy (e.g., via a microwave generator 120 to a waveguide, such as slot waveguide antennas and while maintaining the microwave energy in the MEAD system 100 via microwave reflecting chamber).

The multi-component filter 130 is a good absorber of microwave energy (e.g., microwaves). The desorbed volatiles, which may not be in the same chemical form as they were when the adsorption occurred, are then removed via airflow (e.g., a sweep gas, operating the fan 140). The MEAD system 100 performs desorption (e.g., regeneration) without the multi-component filter 130 being removed for regeneration.

Quantum radiofrequency (RF) physics includes the phenomenon of resonant interaction with matter of electromagnetic radiation in the microwave and RF regions since atoms and molecules can absorb, and thus radiate, electromagnetic waves of various wavelengths. The rotational and vibrational frequencies of the electrons represent a frequency range. The electromagnetic frequency spectrum is usually divided into ultrasonic, microwave, and optical regions. In some embodiments, the microwave region is from 300 megahertz (MHz) to 300 gigahertz (GHz) and encompasses frequencies used for some communication equipment.

The term microwaves or microwave energy may be applied to a broad range of radiofrequency energies particularly with respect to the common heating and/or activating frequencies of about 915 MHz and about 2450 MHz. About 915 MHz is used in industrial heating applications and about 2450 MHz is the frequency of a common household microwave oven. In some embodiments, the MEAD system 100 uses microwave energy (e.g., microwaves) that is radiofrequency energies selected from the range of about 500 to 5000 MHz.

Microwaves lower the effective activation energy for chemical reactions since microwaves can act locally on a microscopic scale by exciting electrons of a group of specific atoms in contrast to normal global heating which raises the bulk temperature. The microscopic interaction is used by polar molecules whose electrons become locally excited leading to high chemical activity. The nonpolar molecules adjacent to such polar molecules are also affected but at a reduced extent. An example is the heating of polar water molecules in a common household microwave oven where the container is of nonpolar material, that is, microwave-passing, and stays relatively cool. In this sense microwaves are often referred to as a form of catalysis when applied to chemical reaction rates.

The MEAD system 100 provides an economically viable device for the microwave cleanup of impure air. The MEAD system 100 contains a multi-component filter 130 for adsorption of impurities that is regenerated in-place with radiofrequency energy in the microwave range by usage of a microwave generator 120 and one or more waveguides (e.g., slot antennas). The housing 110 forms a microwave cavity designed to reflect the microwaves leaving the waveguides into a center section containing the multi-component filter 130.

Microwaves (e.g., microwave energy) are a versatile form of energy that is applicable to enhance chemical reactions since the energy is locally applied by vibrational absorption by nonpolar molecules and does not produce plasma conditions. Reactions that proceed by free-radical mechanisms may be enhanced to higher rates (e.g., their initial equilibrium thermodynamics may be unfavorable).

The multi-component filter 130 may be an excellent microwave energy absorber and may include a wide range of polar impurities that readily interact with radiofrequency energy (e.g., in electron vibrational modes).

The multi-component filter 130 may be used under ambient temperature and pressure conditions. In some embodiments, the multi-component filter 130 includes a metal carbide (e.g., silicon carbide) as a microwave absorbing substrate to enhance catalytic processes.

The microwave excitation of the molecules of the multi-component filter 130, often referred to as microwave catalysis, excites constituents, such as impurities or contaminants including organics, which have been adsorbed on the internal pore surfaces of the multi-component filter 130 and produces a highly reactive condition. Further molecules from the carrier medium, such as a sweep gas (e.g., airflow), are in close proximity or within the surface boundary layer of the surface of the multi-component filter 130 through chemisorption, absorption, adsorption, or diffusion, and additional chemical reactions with these constituents may occur.

The desorption process potentially produces a wide range of chemical compounds since the microwave excited surface of the multi-component filter 130 and possibly the sweep gas molecules react with various decomposition products from the adsorbed constituents. Condensation of collected molecules from the sweep gas can be collected.

In some embodiments, the multi-component filter 130 includes a ceramic filter element that has a hollow space that includes a perforated tube (e.g., a centered perforated stainless steel tube). The space between the perforated tube and the ceramic filter may include pelletized filter material that removes impurities from the airflow. The multi-component filter 130 may be centered at a centerline in the inner volume of the housing 110 that reflects microwaves towards the centerline. One or more waveguides may be disposed in the housing 110 to direct microwaves towards the portions of the inner volume of the housing 110 that includes the multi-component filter 130. Airflow enters the housing 110 (e.g., via an inlet of the housing 110, via an open end of the housing 110), travels through the multi-component filter 130, is purified, and leaves the housing 110 (e.g., via an outlet of the housing).

When the multi-component filter 130 is saturated (e.g., as shown by measurements of impurities via sensors 160, such as a total hydrocarbon analyzer), the microwave generator 120 may be operated (e.g., by the controller 150) to regenerate the microwave generator 120.

In some embodiments, the microwave generator 120 provides microwave energy (e.g., microwaves) from about 850 MHz to about 2450 GHz. The MEAD system 100 may operate continuing cycles of adsorption (e.g., airflow without microwave energy) and desorption (e.g., microwave energy with or without airflow). In some embodiments, the microwave energy is employed at about 1000 watts.

In some embodiments, the MEAD system 100 has an elongated structural microwave cavity with inlet and exit regions configured to reflect microwaves onto a cavity-centered chamber (e.g., cylindrical chamber) that is designed for gas flow with a fixed multi-component filter 130 centered in the chamber. A waveguide (e.g., microwave slot antenna which may be located in the interior volume of the housing 110) may be used to radiate the cavity.

The inlet and exit regions of the housing 110 may be connections for airflow both for purifying the air and regeneration of the multi-component filter 130. The multi-component filter 130 may include at least two penetration depths measured with microwaves of about 2450 MHz. The frequency employed may affect the thickness of the multi-component filter 130 since the bed penetration by microwaves may be frequency dependent and further depend on the mass of the multi-component filter 130. For 2450 MHz microwaves, the penetration thickness (e.g., where the intensity of the RF energy has decreased by e⁻¹) of the multi-component filter 130 may be approximately one inch.

The waveguide (e.g., microwave slot antennas selected from the frequency range of 50 to 5000 MHz) may be capable of flexible operation (e.g., continuous source, pulsed source, cyclic source, periodic source, and combinations thereof). The size and spacing of the slots and the size of the waveguide (e.g., antenna) may be a function of microwave frequency.

In some embodiments, the MEAD system 100 is used to disinfect air (e.g., MEAD system 100 is used an air purification device, an air disinfection device, etc.). In some embodiments, the MEAD system 100 is used to detect a type or quantity of contaminant in the air (e.g., MEAD system 100 is used a contaminant detection device). A small amount of airflow may pass through the MEAD system 100 and sensor data from one or more sensors 160 (e.g., inlet sensor, off-gassing sensor, outlet sensor) can be used to determine whether there is a type or quantity of contaminant. The controller 150 may compare the sensor data (e.g., or differences between sensor data, such as difference between inlet sensor data and outlet sensor data) to threshold values and/or a reference data (e.g., a database of sensor data, a look-up table, etc.) to determine whether there is a type or quantity of contaminant in the air. Responsive to determining there is a type or quantity of contaminant in the air, the controller 150 may cause a corrective action (e.g., provide an alert, cause one or more other MEAD systems 100 to have a particular operation to disinfect the air, etc.).

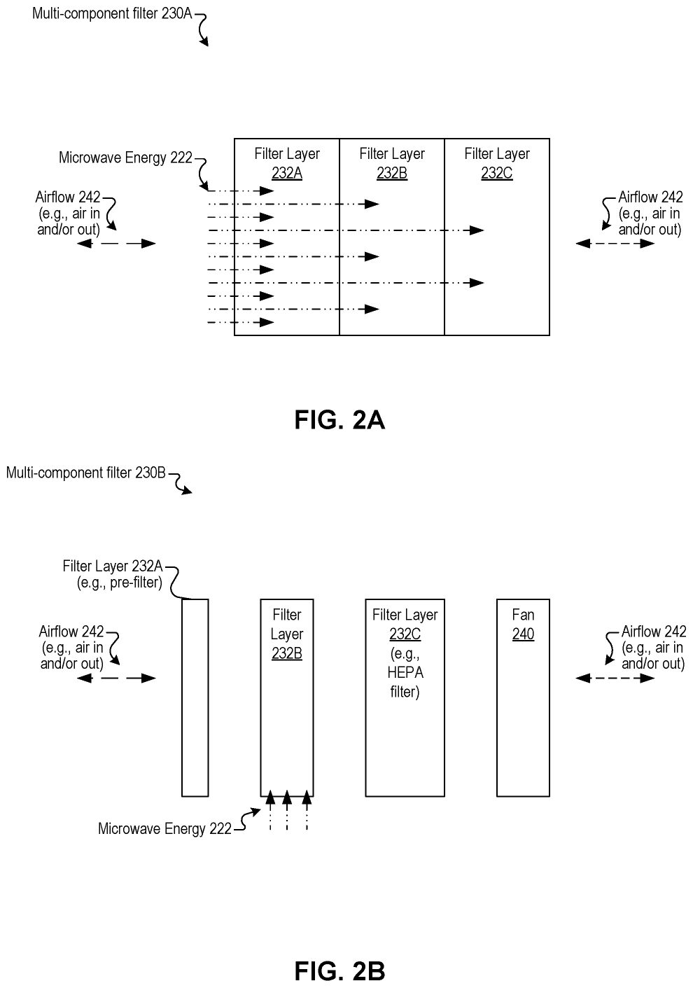

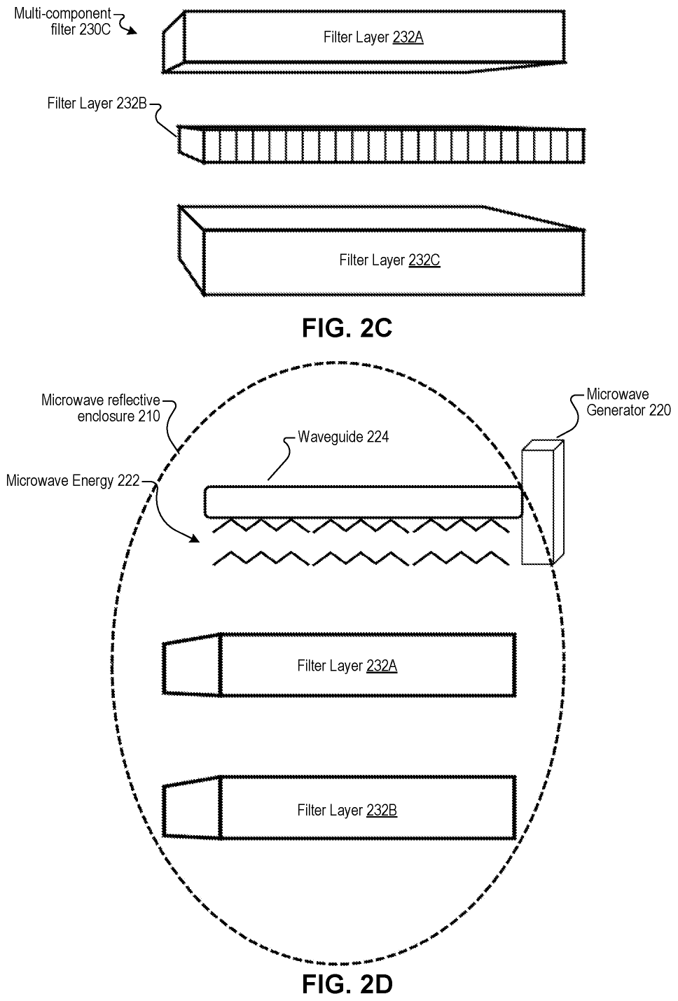

FIGS. 2A-D illustrate multi-component filters 230 of MEAD systems 200, according to certain embodiments. Components of FIGS. 2A-D that have similar reference numbers as components in FIGS. 1A and/or B may have similar or the same structure and/or functionality. Multi-component filters 230 of FIGS. 2A-D may include at least some of the same structure and/or functionality of the multi-component filter 130 of FIGS. 1A and/or 1B. The MEAD systems 200 of FIGS. 2A-D may have at least some of the same structure and/or functionality of the MEAD system 100 of FIGS. 1A and/or 1B.

In some embodiments, the MEAD system 200 provides airflow (e.g., clean air, disinfected air) to an indoor space (e.g., building, office, home, factory, healthcare facility, restaurant, etc.). The MEAD system 200 may be located inside the indoor space (e.g., as a stand-alone device). In some embodiments, the MEAD system 200 is configured to be removably placed on a surface, such as a floor, table, shelf, furniture, etc. In some embodiments, the MEAD system 200 is located inside ducting, piping, HVAC system, etc. that provides airflow to and/or from an indoor space. In some embodiments, the MEAD system 200 is integrated into an HVAC unit (e.g., furnace, air handler, roof top unit (RTU), heat pump, etc.). In some embodiments, the MEAD system 200 is retrofit to an HVAC unit.

The MEAD system 200 receives airflow 242 (e.g., air in, contaminated air) from the indoor space and provides airflow 242 (e.g., air out, clean air) back into the indoor space. In some embodiments, the airflow 242 (e.g., contaminated air) that enters the MEAD system 200 includes one or more of hairs, fibers, pathogens, moisture droplets, particles, VOCs, other gases. The airflow 242 (e.g., contaminated air) flows through the multi-component filter 230. The multi-component filter 230 includes multipole components (e.g., a linear stack of two or more filter layers 232, a heterogeneous mix of filter materials, etc.). In some embodiments, filter layer 232A (e.g., a first filter layer) removes (e.g., destroys, off-gases) large contaminants, such as hairs, fibers, larger moisture droplets, larger particles, etc. Filter layer 232B (e.g., second filter layer) removes (e.g., destroys, off-gases) smaller contaminants, such as pathogens, smaller moisture droplets, smaller particles, VOCs, gases (e.g., gas contaminants), etc. Filter layer 232C removes (e.g., destroys, off-gases) remaining contaminants, such as pathogens, smaller moisture droplets, and smaller particles. In some embodiments, when microwave energy 222 is applied, a first portion of the multi-component filter 230 (e.g., the first filter layer, filter layer 232A, metal oxide, microwave-absorbing layer, foam cylindrical silicon carbide filter, etc.) gets hot (e.g., extremely hot) and/or activated and oxidizes the trapped contaminants. A second portion of the multi-component filter 230 (e.g., the second filter layer, filter layer 232B, molecular sieve, zeolite layer) also heats and/or is activated and destroys the adsorbed contaminants. In some embodiments, any organic material caught in a third portion of the multi-component filter 230 (e.g., third filter layer, filter layer 232C, HEPA filter, high-temperature cylindrical HEPA filter) is also heated and/or activated and oxidizes contaminants. The microwave energy 222 destroys contaminants and keeps filter layers clean. In some embodiments, one or more portions of the multi-component filter 230 (e.g., third filter layer, filter layer 232C, HEPA filter) does not absorb microwave energy 222.

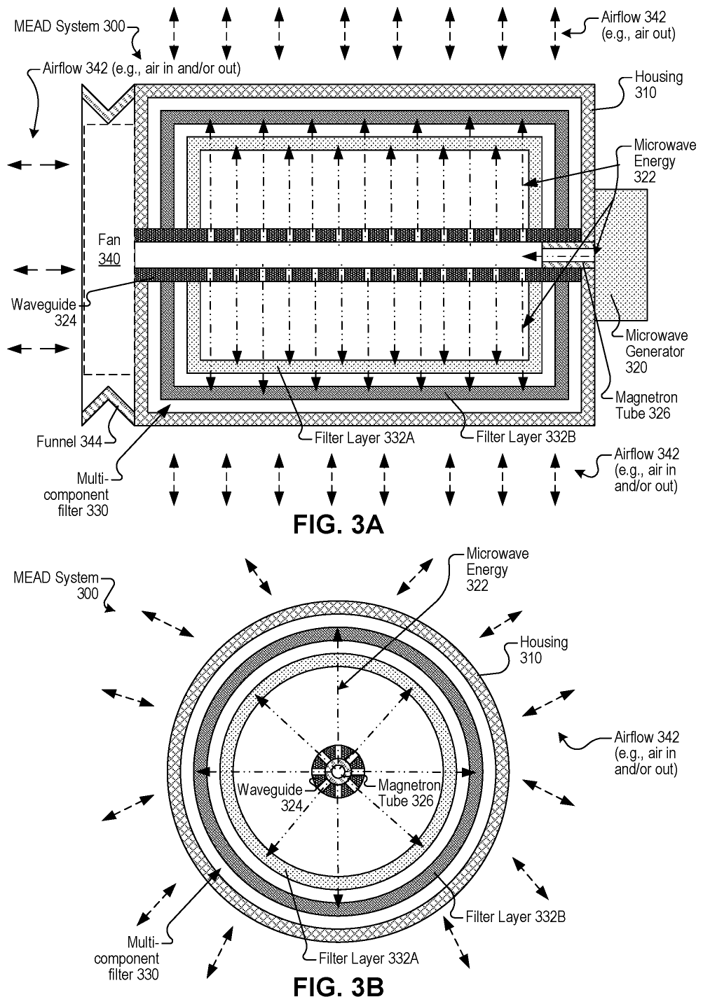

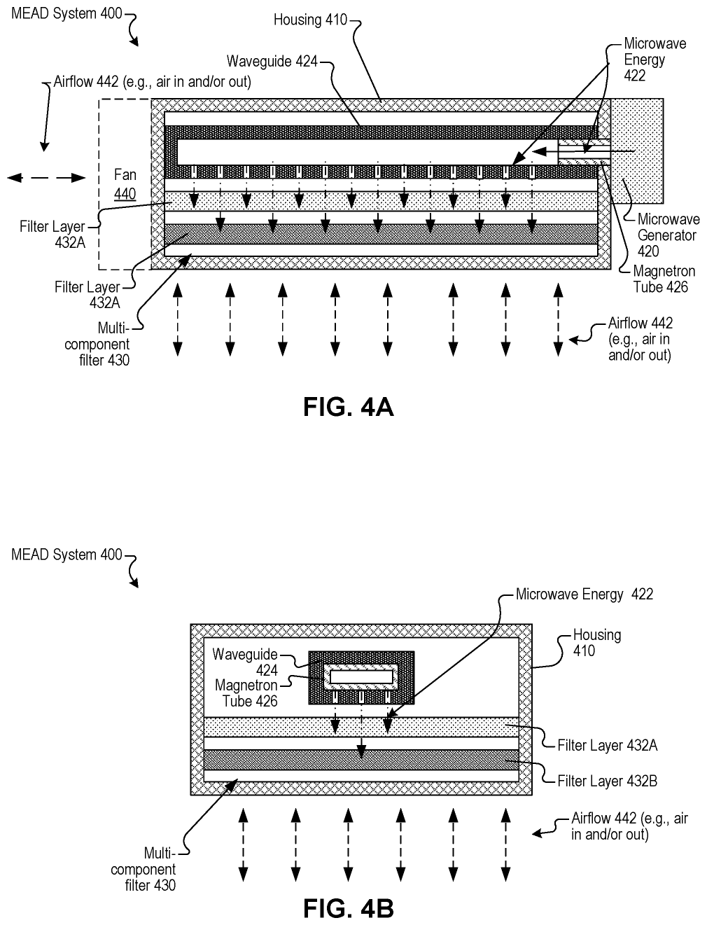

In some embodiments, the multi-component filter 230 is cylindrical (e.g., see FIGS. 3A-B). In some embodiments, the multi-component filter 230 is flat (e.g., see FIGS. 4A-B). In some embodiments, the order of the filter layers 232 (e.g., HEPA filter first, last, or middle) is adjusted. In some embodiments, the distribution of microwave energy 222 is controlled depth-wise based on design and composition of the multi-component filter 230 (e.g., types of filter materials, depth of filter layers 232, order of filter layers 232, etc.). Since the microwave energy 222 cleans the multi-component filter 230, thinner filter layers and better filtering media can be used.

Referring to FIG. 2A, the multi-component filter 230A includes filter layers 232A-C. A first portion of microwave energy 222 is provided into filter layer 232A, a second portion (e.g., that is less than the first portion) of the microwave energy 222 is provided into the filter layer 232B, and a third portion (e.g., that is less than the second portion) of the microwave energy 222 is provided into the filter layer 232C. Airflow 242 enters the multi-component filter 230A at the filter layer 232A, passes through the filter layer 232B, and exits the multi-component filter 230A at the filter layer 232C.

Referring to FIG. 2B, the multi-component filter 230B includes filter layer 232A (e.g., pre-filter, removable filter), filter layer 232B (e.g., metal oxide) that receives microwave energy 222, and a filter layer 232C (e.g., HEPA filter). A fan 240 (e.g., fan 140 of FIGS. 1A and/or B) causes airflow 242 through the multi-component filter 230B. In some embodiments, the fan 240 pulls airflow 242 from outside of the housing 110, through the filter layer 232A, then through the filter layer 232B, then through the filter layer 232C, then through the fan 240, and then causes the airflow 242 to exit the housing 110. In some embodiments, the filter layer 232A (e.g., pre-filter) has a smaller depth (e.g., distance the airflow 242 flows through) than the filter layer 232B (e.g., microwave-absorbing filter layer, metal oxide) and the filter layer 232B has a smaller depth than the filter layer 232C (e.g., HEPA filter).

The MEAD system 200 is a microwave-activated filter system that collects and destroys a variety of contaminants, such as a variety of microbes and VOCs. The MEAD system 200 (e.g., multi-component filter 230) destroys contaminants via microwave energy effects on cell structures, activation of antimicrobial properties of materials in the multi-component filter 230, and/or heating that kills microbes. The microwave applicator (e.g., microwave generator, magnetron, and/or waveguide) controls distribution of microwave energy to the multi-component filter 230.

Referring to FIG. 2C, the multi-component filter 230C (e.g., multilayer filter) includes filter layer 232A (e.g., metal oxide, metal oxide impregnated with insulating and high temperature media for VOC destruction), filter layer 232B (e.g., zeolite layer, molecular sieve, microwave reactive and conductive layer that causes heating and/or oxidation reactions), and a filter layer 232C (e.g., HEPA filter layer to capture dust particles down to minus 2.5 microns).

Referring to FIG. 2D, the multi-component filter 230D includes filter layer 232A (e.g., metal oxide, flat microwave-reactive filter) and filter layer 232B (e.g., HEPA filter). The multi-component filter 230D is disposed within a microwave reflective enclosure 210 (e.g., housing 110 of FIGS. 1A and/or B). In some embodiments, a microwave generator 220 (e.g., microwave generator 120 of FIGS. 1A and/or B) is coupled to (e.g., at least partially disposed within, disposed proximate) the microwave reflective enclosure 210. In some embodiments, the microwave generator 220 is coupled to or includes one or more magnetrons.

The microwave generator 220 is coupled (e.g., attached, fluidly coupled to) a waveguide 224 (e.g., slotted rectangular waveguide, circular slotted leaky waveguide, cylindrical slotted waveguide, quartz tube, etc.). The waveguide 224 provides uniformity in directing the microwave energy 222. The waveguide 224 provides low reflectivity of the microwave energy 222. The waveguide 224 is hollow to receive the microwave energy 222 generated by the microwave generator 220. The waveguide 224 directs the microwave energy 222 towards the multi-component filter 230D to heat and/or activate at least a portion of the multi-component filter 230 (e.g., filter layer 232A) to remove contaminants from the airflow 242 (e.g., destroy, oxidize, off-gas, etc. contaminants from the airflow 242 that were trapped by the multi-component filter 230D).

In some embodiments, the microwave generator 220, magnetron, and/or waveguide 224 are tailored to the multi-component filter 230 configuration (e.g., planar, cylindrical, or other shape of waveguide 224). In some embodiments, the microwave energy 222 (e.g., microwaves) is contained in the unit (e.g., no leakage, no safety issues) via one or more components of the MEAD system 200 (e.g., the microwave reflective enclosure 210). In some embodiments, one or more sensors 160 and the controller 150 are used to detect leakage and provide a corrective action (e.g., remedy, shutdown, alert, etc.). In some embodiments, the controller 150 provides software alerts. In some embodiments, the controller 150 allows the MEAD system 200 to be controlled from a mobile device and provides alerts (e.g., notifications) to the mobile device. In some embodiments, the MEAD system 200 is integrated into a smart home environment and/or into larger HVAC systems.

The microwave energy 222 causes oxidation and/or other reactions (e.g., by heating the multi-component filter 230 to destroy trapped contaminants) which provides an off gas. The sensors 160 and controller 150 are used to one or more of determine composition of contaminants (e.g., potential dangerous but invisible pathogens) in incoming airflow, confirm destruction of the contaminants, determine efficiency of the MEAD system, provide other user information about indoor air safety and quality, provide information about effectiveness of the MEAD system 200, etc.

In some embodiments, the sensors 160 and controller 150 are used to provide a mode of operation (e.g., always on or on/off, frequency of on/off). The MEAD system 200 provides a long-life filter. In some embodiments, the MEAD system 200 receives airflow 242 (e.g., sucks in air) via an upper portion of the MEAD system 200 and provides airflow 242 (e.g., clean air) via one or more side portions or a lower portion of the MEAD system 200. In some embodiments, the MEAD system 200 has a motor (e.g., fan 240) that is designed for super quiet operation in microwave environment. In some embodiments, the MEAD system 200 has chambers (e.g., interior volume formed by the housing 110) that reflect microwave energy 222 for better uniform distribution.

In some embodiments, real-time data is collected from multiple MEAD systems 200 (e.g., thousands or millions of MEAD systems 200 across geographies) to track progression of an infection or pollution wave, detect pollution sources, etc. In some embodiments, a server device uses the collected data (e.g., off-gas analysis) from multiple MEAD systems 200 to determine regional (e.g., local, national, etc.) patterns.

In some embodiments, the MEAD system 200 does not include carbonaceous material (e.g., activated carbon, char, soot, pyrolytic carbon, carbon black, activated charcoal, etc.). In some embodiments, any carbonaceous material in the MEAD system 200 is located to not receive microwave energy 222 or to receive less than a threshold amount of microwave energy 222. In some embodiments, a microwave reflective enclosure is located between the microwave energy 222 (e.g., waveguide 224, etc.) and the carbonaceous material. In some embodiments, the carbonaceous material is the furthest or one of the furthest filter layers from the microwave energy 222 (e.g., waveguide 224, etc.). In some embodiments, the multi-component filter 230 is used to collect hazardous materials (e.g., contaminants). In some embodiments, the multi-component filter 230 is regenerated (e.g., destroying of the contaminants) via the microwave energy 222. In some embodiments, the multi-component filter 230 is a tubular design with a slotted waveguide located in the center of the multi-component filter 230 with microwave reflection layer surrounding the multi-component filter 230 (e.g., see FIGS. 3A-B). In some embodiments, the multi-component filter 230 is a horizontal filter located using a slotted waveguide with microwave reflective layer surrounding the multi-component filter 230 (e.g., see FIGS. 4A-B).

FIGS. 3A-B are cross-sectional views of a MEAD system 300 (e.g., MEAD system 100 of FIGS. 1A and/or B), according to certain embodiments. Components of FIGS. 3A-B that have similar reference numbers as components in one or more of FIGS. 1-2D may have at least some of the same structure and/or functionality. FIG. 3A is a cross-sectional view length-wise of MEAD system 300 and FIG. 3B is a cross-sectional view width-wise of the MEAD system 300.

In some embodiments, the MEAD system 300 is a device (e.g., a stand-alone device, a device that can be installed in a system, a device that can be installed in ductwork, etc.). In some embodiments, the MEAD system 300 is substantially cylindrical.

In some embodiments, the MEAD system 300 includes a waveguide 324 that is routed through a central portion of the MEAD system 300 (e.g., along a longitudinal axis of the MEAD system 300, along a longitudinal axis of housing 310). In some embodiments, the waveguide 324 is cylindrical and slotted.

A first distal end of the MEAD system 300 may include a fan 340 disposed within a funnel 344 that is coupled to the housing 310. A second distal end of the MEAD system includes a microwave generator 320 coupled to the housing 310.

In some embodiments, the microwave generator 320 is coupled to the waveguide 324 via a magnetron tube 326. In some embodiments, the magnetron tube 326 has an outside perimeter (e.g., outer circumference) that is configured to fit within the inside diameter (inner circumference) of the waveguide 324. A housing 310 disposed around the waveguide 324. A multi-component filter 330 is disposed between the housing 310 and the waveguide 324. In some embodiments, the multi-component filter 330 is substantially a hollow cylinder. In some embodiments, the multi-component filter 330 includes two or more filter layers 332 (e.g., filter layers 332A-B). In some embodiments, the filter layers 332 contact each other. In some embodiments, the filter layers 332 are spaced apart. In some embodiments, filter layer 332A is a tubular microwave-reactive filter media. In some embodiments, filter layer 332B is a tubular HEPA filter with microwave reflective screening.

The microwave generator 320 generates microwave energy 322 that is channeled by the magnetron tube 326 into the waveguide 324 that directs the microwave energy 322 towards the multi-component filter 330. The fan 340 provides airflow 342 into the housing 310 to cool the microwave generator 320 and to pass through the multi-component filter 330 and then through the housing 310. In some embodiments, fan 340 (e.g., ventilation fan that turns off during heating of the multi-component filter 330) provides airflow into the housing 310 and a second fan (e.g., cooling fan disposed in housing of the microwave generator 320) provides airflow to cool the microwave generator 320 (e.g., magnetron). Contaminants from the airflow 342 become trapped on the multi-component filter 330 and the microwave energy 322 causes the multi-component filter 330 to heat and/or activate to destroy the contaminants. In some embodiments, the microwave energy 322 is applied in a 360 degree pattern (e.g., around the cylindrical perimeter of the waveguide 324).

FIGS. 4A-B are cross-sectional views of a MEAD system 400 (e.g., MEAD system 100 of FIGS. 1A and/or B), according to certain embodiments. Components of FIGS. 4A-B that have similar reference numbers as components in one or more of FIGS. 1-3B may have at least some of the same structure and/or functionality. FIG. 4A is a cross-sectional view length-wise of MEAD system 400 and FIG. 4B is a cross-sectional view width-wise of the MEAD system 400.

In some embodiments, the MEAD system 400 is a device (e.g., a stand-alone device, a device that can be installed in a system, a device that can be installed in ductwork, etc.). In some embodiments, the MEAD system 400 is substantially a rectangular prism (e.g., opposing sides of the housing 410 are substantially parallel).

In some embodiments, the MEAD system 400 includes a waveguide 424 that is routed through the MEAD system 400 (e.g., parallel to a longitudinal axis of the MEAD system 400, parallel to a longitudinal axis of housing 410). In some embodiments, the waveguide 424 is a hollow rectangular prism and slotted (e.g., with slots directed towards the multi-component filter).

In some embodiments, a first distal end of the MEAD system 400 includes a fan 440 (e.g., disposed within a funnel that is coupled to the housing 410). A second distal end of the MEAD system includes a microwave generator 420 coupled to the housing 410.

In some embodiments, the microwave generator 420 is coupled to the waveguide 424 via a magnetron tube 426. In some embodiments, the magnetron tube 426 has an outside perimeter that is configured to fit within the inside diameter of the waveguide 424. A housing 410 disposed around the waveguide 424. A multi-component filter 430 is disposed between the housing 410 and the waveguide 424. In some embodiments, the multi-component filter 430 is substantially flat and is located between one side of the waveguide 424 and the housing 410. In some embodiments, the multi-component filter 430 includes two or more filter layers 432 (e.g., filter layers 432A-B). In some embodiments, the filter layers 432 contact each other. In some embodiments, the filter layers 432 are spaced apart.

The microwave generator 420 generates microwave energy 422 that is channeled by the magnetron tube 426 into the waveguide 424 that directs the microwave energy 422 towards the multi-component filter 430. The fan 440 may provide airflow 442 into the housing 410 to cool the microwave generator 420 and to pass through the multi-component filter 430 and then through the housing 410. In some embodiments, fan 440 (e.g., ventilation fan that turns off during heating of the multi-component filter 430) provides airflow into the housing 410 and a second fan (e.g., cooling fan disposed in housing of the microwave generator 420) provides airflow to cool the microwave generator 420 (e.g., magnetron). Contaminants from the airflow 442 become trapped on the multi-component filter 430 and the microwave energy 422 causes the multi-component filter 430 to heat and/or activate to destroy the contaminants.

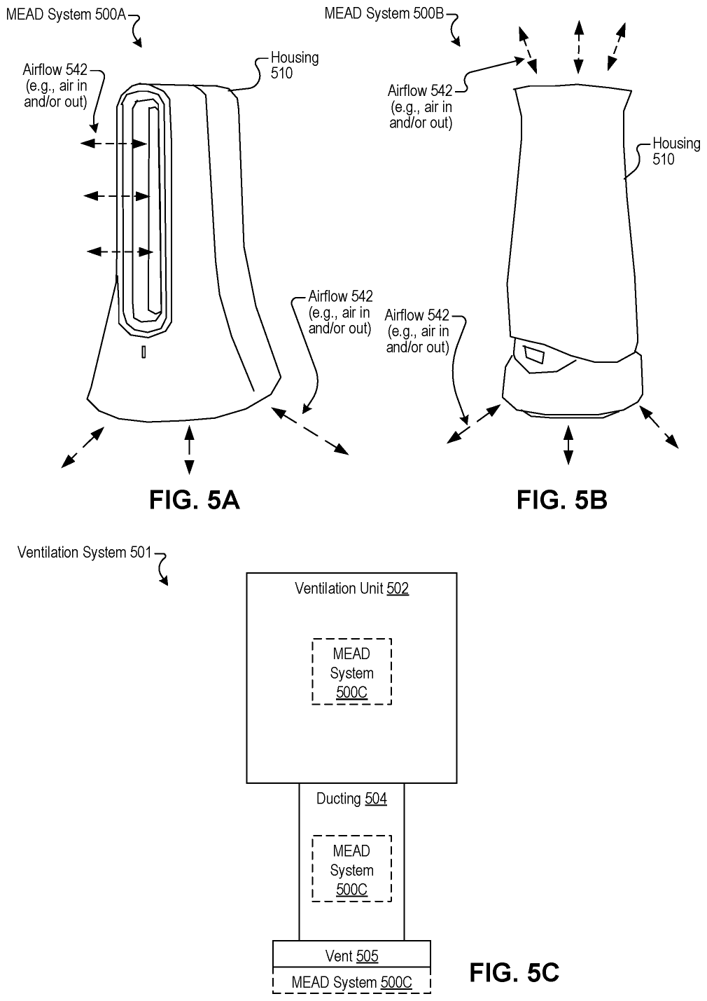

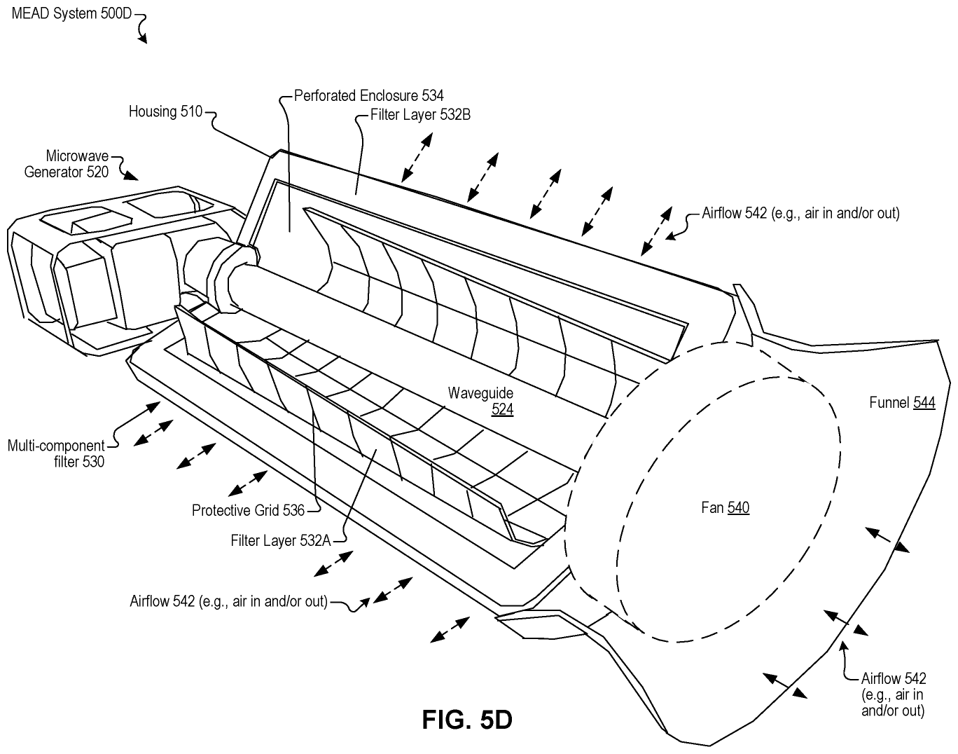

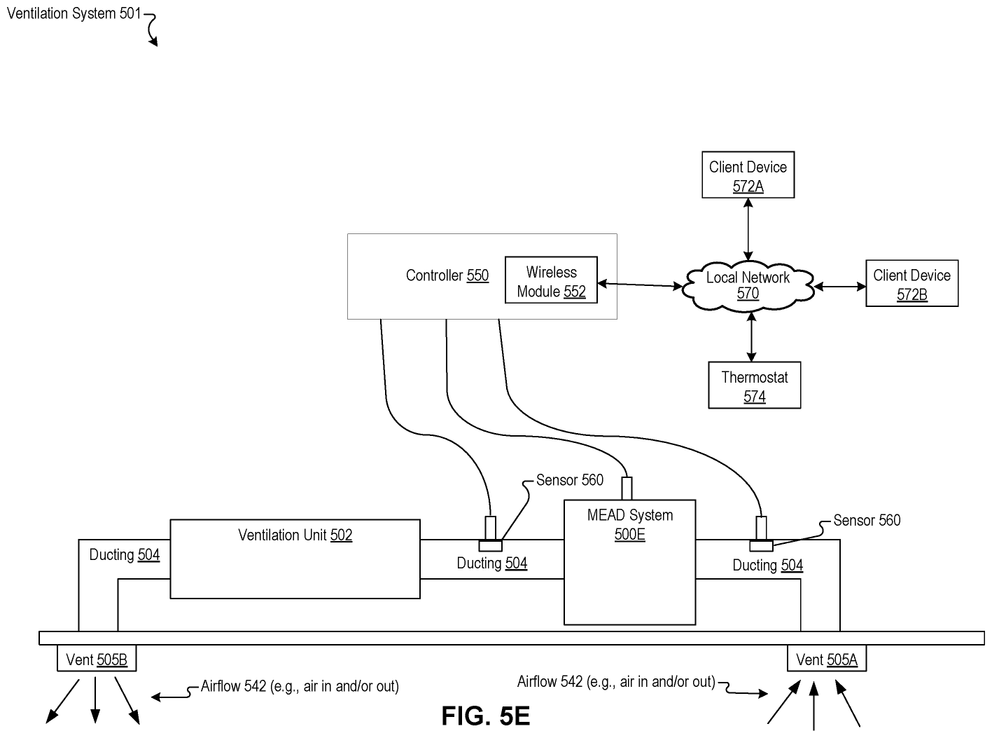

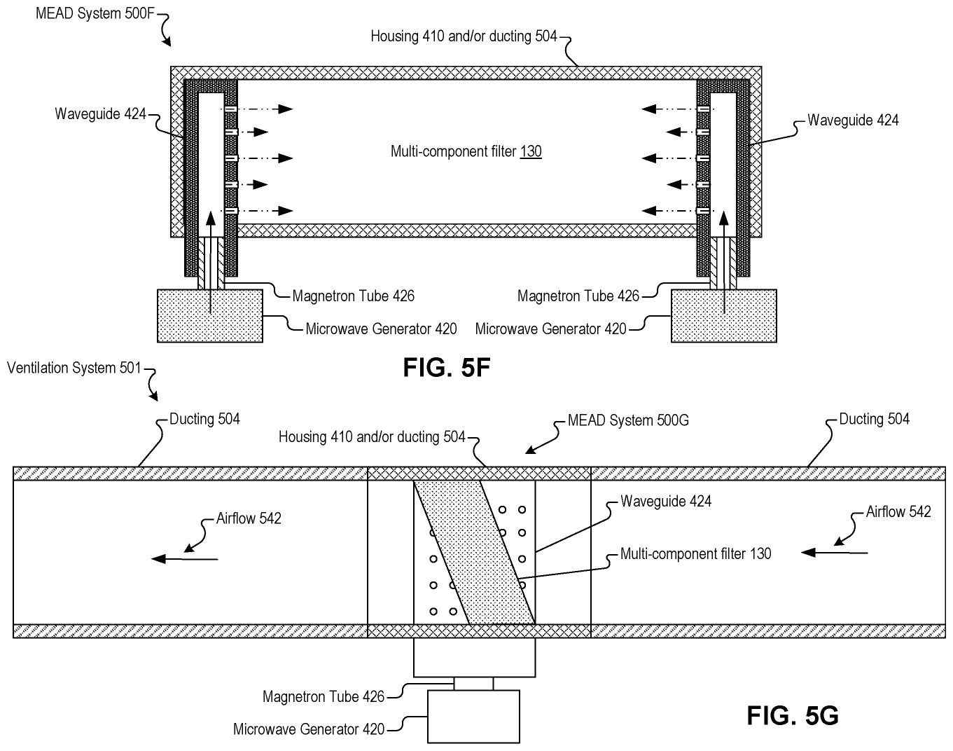

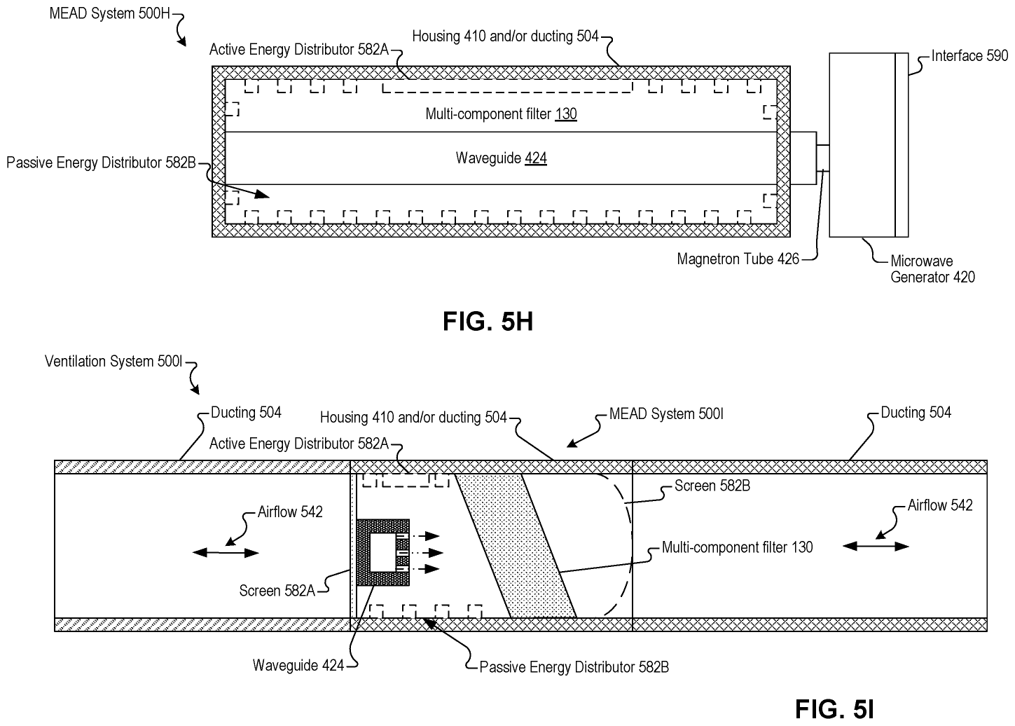

FIGS. 5A-I illustrate MEAD systems 500A-I, according to certain embodiments. Components of FIGS. 5A-I that have similar reference numbers as components in one or more of FIGS. 1-4B may have at least some of the same structure and/or functionality.

Referring to FIG. 5A, MEAD system 500A has a housing 510 that houses one or more of a microwave generator, multi-component filter, fan, controller, sensors, waveguide, and/or magnetron tube. In some embodiments airflow 542 enters the housing 510 via one or more openings proximate a lower surface of the housing 510. In some embodiments, the housing 510 forms an opening on a front side of the housing 510 and airflow 542 exits the housing 510 via the opening. In some embodiments, the housing 510 includes a user interface (e.g., light emitting diode (LED), touch screen, buttons, and/or the like). In some embodiments, the MEAD system 500A is used as a cooling device (e.g., convection cooling) for user comfort by running the fan even when the microwave generator is not generating microwave energy. In some embodiments, the MEAD system 500A has a user interface includes options to actuate the airflow (e.g., at different flowrates, such as high, medium, and low).

Referring to FIG. 5B, MEAD system 500B may be similar to MEAD system 500A, but instead of directing the airflow 542 out through an opening formed in a front of the housing 510, MEAD system 500B directs the airflow 542 out through openings formed in an upper surface of the housing 510.

In some embodiments, a first set of surfaces of MEAD system 500A and/or 500B form openings (e.g., perforated, slotted, etc.) for receiving airflow into the housing 510 and a second set of surfaces of MEAD system 500A and/or 500B form openings for providing airflow out of the housing 510.

Referring to FIG. 5C, one or more MEAD systems 500C may be located in conjunction with (e.g., inside, proximate to) a ventilation system 501. A ventilation system 501 may be a building ventilation system, a vehicle ventilation system, etc. A ventilation system 501 may include a ventilation unit 502 (e.g., HVAC unit, building ventilation unit, vehicle ventilation unit, etc.). The ventilation system 501 may include ducting 504 coupled to the ventilation unit 502. Ducting 504 may include one or more of supply air ducting, return air ducting, outside air ducting, piping, and/or the like. The ventilation system 501 may include one or more vents 505. Vents 505 may be used to control air flow direction, control air flow rate, control amount of air flow, balance air, etc. Vents 505 may include one or more of a grille (e.g., intake, exhaust), a register (e.g., with an adjustable damper, provide airflow into a room, control airflow direction, etc.), a diffuser (e.g., including dampers, provide airflow into a room), etc.

In some embodiments, the MEAD system 500C is disposed inside the airflow within the HVAC unit 502 (e.g., before or after the heat exchanger and/or cooling coil). By disposing the MEAD system 500C before the heat exchanger and/or cooling coil, the MEAD system 500C may prevent contaminants from damaging or soiling the heat exchanger and/or cooling coil. By locating the MEAD system 500C after the heat exchanger and/or cooling coil, the microwave generator of the MEAD system 500C be operated less often (e.g., other components of the HVAC unit 502 remove some of the contaminants from the airflow).

In some embodiments, the HVAC unit 502 provides the airflow through the MEAD system 500C (e.g., the MEAD system 500C may not include a fan).

In some embodiments, the one or more MEAD systems 500C in the ventilation system 501 provide minimal pressure drop to the ventilation system 501. In some embodiments, the one or more MEAD systems 500C in the ventilation system 501 provide a total pressure drop of about 0 to about 0.5 inches of water gauge (in wg) to the ventilation system 501. In some embodiments, the one or more MEAD systems 500C in the ventilation system 501 provide a total pressure drop of less than about 2 in wg to the ventilation system 501. In some embodiments, the one or more MEAD systems 500C in the ventilation system 501 provide a total pressure drop of about 1 to about 2 in wg to the ventilation system 501. In some embodiments, the one or more MEAD systems 500C in the ventilation system 501 provide a total pressure drop of less than about 1 in wg to the ventilation system 501. In some embodiments, the one or more MEAD systems 500C in the ventilation system 501 provide a total pressure drop of less than about 0.5 in wg to the ventilation system 501. In some embodiments, the one or more MEAD systems 500C in the ventilation system 501 provide a total pressure drop of less than about 0.25 in wg to the ventilation system 501.

In some embodiments, the MEAD system 500C is designed as a filter box to replace factory filters prior to heating/cooling coils of a ventilation system 501 (e.g., HVAC system). Referring to FIG. 5D, MEAD system 500C may not have a fan and may interface with the control system (e.g., HVAC control system) of ventilation system 501.

In some embodiments, the MEAD system 500C is configured to destroy allergens and/or pathogens while minimizing pressure drop in the ventilation system 501 (e.g., has stripped-down functionality, without capturing all other types of particles). In some embodiments, the MEAD system 500C uses a desiccant filter (e.g., without a HEPA filter). In some embodiments, the MEAD system 500C uses a desiccant filter and/or a zeolite filter. In some embodiments, the MEAD system 500C is configured to remove (e.g., destroy, off-gas) one or more of pathogens, smaller moisture droplets, smaller particles, VOCs, gases (e.g., gas contaminants, etc. in some embodiments, the MEAD system 500C is configured to destroy contaminants (e.g., via microwave energy and/or material properties of a filter of MEAD system 500C, etc.).

In some embodiments, ventilation system 501 includes multiple MEAD systems 500C (e.g., instead of a single centralized MEAD system 500C), where each MEAD system 500C is located in a corresponding distribution points of ducting 504, vent 505, or in a room. In some embodiments, each MEAD system 500C in ventilation system 501 has a microwave energy generator. The MEAD systems 500C are controlled (e.g., by a processing device) so that a threshold amount of energy (e.g., electrical current) is not exceeded. In some examples, the MEAD systems 500C are controlled so that the microwave energy generator of only one MEAD system 500C is operated at a time.

In some embodiments, one or more MEAD systems 500C are disposed inside the ducting 504 of ventilation system 501. In some embodiments, MEAD systems 500C and pressure balancing devices (e.g., dampers, louvers, vents 505, ducting 504, etc.) to manage air flow through the ventilation system 501. In some embodiments, one or more MEAD systems 500C are disposed inside the return air ducting of the ventilation system 501 (e.g., after the return air ducting filter such as a furnace filter, to destroy contaminants coming from the rooms). In some embodiments, one or more MEAD systems 500C are disposed inside the supply air ducting of the ventilation system 501 (e.g., to destroy contaminants coming from the ventilation unit 502, outside air, etc.).

In some embodiments, the MEAD system 500C is disposed in conjunction with the vent 505. In some examples, the MEAD system 500C is disposed between the vent 505 and the ducting 504. In some examples, the vent is disposed between the ducting 504 and the MEAD system 500C. In some examples, the vent is disposed in the vent 505.

Referring to FIG. 5D, FIG. 5D is a partial cross-section of MEAD system 500D. The MEAD system 500D includes a housing 510, a funnel 544 (e.g., exhaust funnel) attached to the housing 510, a fan 540 (e.g., air exhaust fan) disposed in the funnel 544, a microwave generator 520 (e.g., microwave magnetron unit) coupled (e.g., attached) to the housing 510. Inside of the housing 510, the MEAD system 500D includes a waveguide 524 (e.g., leaky waveguide), a filter layer 532A (e.g., silicon carbide (SiC) layer), a protective grid 536 (e.g., SiC protective grid) of the filter layer 532A, a filter layer 532B (e.g., HEPA filter), and a perforated enclosure 534 (e.g., HEPA filter perforated enclosure) of the filter layer 532B.

In some embodiments, the airflow 542 enters the housing 510 via the funnel 544 and exits the housing 510 after passing through the filter layer 532B (e.g., via the cylindrical outer surface area of the housing 510). In some embodiments, the airflow 542 enters the housing 510 via the sidewalls of the housing 510 (e.g., the cylindrical outer surface area of the housing 510, proximate the filter layer 532B) and exits the housing 510 via the funnel 544. In some embodiments, the airflow 542 is alternated between entering the housing via the funnel 544 and entering the housing via the sidewalls of the housing 510. In some embodiments, the HEPA filter is closer to the housing 510 (e.g., is the outer filter layer) and the SiC layer is closer to the waveguide 524 (e.g., is the inner filter layer). In some embodiments, the HEPA filter is closer to the waveguide 524 (e.g., is the inner filter layer) and the SiC layer is closer to the housing 510 (e.g., is the outer filter layer).

FIG. 5E is a diagram of a ventilation system 501 including a MEAD system 500E, according to certain embodiments.

In some embodiments, a ventilation system 501 includes a ventilation unit 502. The ventilation unit 502 may include a variable refrigerant volume (VRV) unit, a heat pump, a furnace, a roof-top unit (RTU), multi-split type air conditioner, an air handler, etc. The ventilation unit 502 may include a fan to provide airflow 542 through vents 505, ducting 504, MEAD system 500E, and ventilation unit 502. In some embodiments, an air handler provides the airflow 542 and the ventilation unit 502 includes one or more of a heating component to heat the airflow 542, a cooling component to cool the airflow 542, a flowrate component (e.g., damper) to control (e.g., increase or decrease) the airflow 542, etc.

The ventilation system 501 may include one or more vents 505 (e.g., diffusers, grilles, registers, etc.). Vent 505A may be an intake (e.g., return) to the ventilation system 501 and vent 505B may be an outlet (e.g., supply) of the ventilation system 501. The ventilation system 501 may include ducting 504 that connects the ventilation unit 502, the vents 505, and a MEAD system 500E. For example, a first segment of ducting 504 may connect the vent 505A with the MEAD system 500E, a second segment of ducting 504 may connect the MEAD system 5003 with the ventilation unit 502, and a third segment of ducting may connect the ventilation unit 502 with vent 505B. In some embodiments, MEAD system 500E is used instead of a filter box.

Ventilation system 501 may include a controller 550. The controller 550 may be coupled (e.g., via wired communication, via wireless communication) with the MEAD system 500E and one or more sensors 560. The one or more sensors 560 may be disposed in one or more of ducting 504, ventilation unit 502, MEAD system 500E, vent 505, the room being supplied by ventilation system 501, etc.

The controller 550 may include a wireless module 552 to communicate with a local network 570. The controller 550 may communicate via the local network 570 with one or more client devices 572, thermostat 574, and/or the like.

Local network 570 may be a computing network that provides one or more communication channels between components (e.g., MEAD systems, controller 550, client device 572, thermostat 574, etc.). In some examples, local network 570 is a peer-to-peer network that does not rely on a pre-existing network infrastructure (e.g., access points, switches, routers) and controller 550 replaces the networking infrastructure to route communications between the components. Local network 570 may be a wireless network that is self-configuring and enables components to contribute to local network 570 and dynamically connect and disconnect from local network 570 (e.g., ad hoc wireless network). In some examples, local network 570 is a computing network that includes networking infrastructure that enables components to communicate with other components. The local network 570 may or may not have access to the public network (e.g., internet). For example, an access point or device that may function as an access point to enable components to communicate with one another without providing internet access. In some embodiments, the local network 570 includes or provides access to a larger network such as one or more of a public network that provides components with access to each other (e.g., other publically available computing devices) or a private network that provides components access to each other (e.g., other privately available computing devices). In some embodiments, local network 570 includes or provides access to one or more Wide Area Networks (WANs), Local Area Networks (LANs), wired networks (e.g., Ethernet network), wireless networks (e.g., an 802.11 network or a Wi-Fi® network), cellular networks (e.g., a Long Term Evolution (LTE) network), radar units, transmission antenna, reception antenna, microwave transmitter, microwave receiver, sonar devices, Lidar devices, routers, hubs, switches, server computers, cloud computing networks, and/or a combination thereof. In some embodiments, local network 570 is based on any wireless or wired communication technology and may connect a first component directly or indirectly (e.g., involving an intermediate device, such as an intermediate component) to a second component. The wireless communication technology may include Bluetooth®, Wi-Fi®, infrared, ultrasonic, or other technology. The wired communication may include universal serial bus (USB), Ethernet, RS 232, or other wired connection. The local network 570 may be an individual connection between two components or may include multiple connections.

In some embodiments, the client device 572 includes a computing device such as Personal Computers (PCs), laptops, mobile phones, smart phones, tablet computers, netbook computers, gateway device, etc. Client device 572 includes an operating system that allows users to one or more of generate, view, or edit data (e.g., settings of MEAD system, corrective actions associated with MEAD systems, etc.).

In some embodiments, the controller 550 includes one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc. In some embodiments, the controller 550 is an input/output (I/O) daughter card. The controller 550 may determine one or more of pressure data, temperature data, carbon dioxide (CO₂) data, relative humidity data, VOC data, particulate matter that is about 2.5 microns or less in diameter (PM2.5), particulate matter that is about 10 microns or less in diameter (PM10), and/or the like. The controller 550 may receive sensor data from one or more sensors 560 and may cause the sensor data and/or an alert to be displayed (e.g., via client device 572). In some embodiments, the controller 550 receives setpoints (e.g., particulate matter setpoints, etc.) via local network 570 (e.g., from client device 572, from thermostat 574) and causes the MEAD system 500E to meet the setpoints (e.g., meet the particulate matter setpoints, etc.).

In some embodiments, the MEAD system 500E is disposed within the ducting 504. In some embodiments, MEAD system 500E is coupled to the ducting 504 so that the airflow 542 through the ducting 504 goes through the MEAD system 500E.

FIG. 5F is a cross-sectional view of a MEAD system 500F. MEAD system 500F may include a multi-component filter 130 that has one dimension (e.g., width) that is greater than another dimension (e.g., height). For example, ducting 504 may be about 4 feet wide by 12 inches tall and the MEAD system 500F may have similar dimensions. The MEAD system 500F may include multiple waveguides 424 to provide microwave energy to the multi-component filter 130 (e.g., to distribute heat substantially equally over longer width). In some embodiments, a first waveguide 424 is disposed proximate the left side of the MEAD system 500F and a second waveguide 424 is disposed proximate the right side of the MEAD system 500F. In some embodiments, a MEAD system 500F includes multiple waveguides 424 including a waveguide 424 is disposed proximate the left side of the MEAD system 500F, a waveguide 424 is disposed proximate the right side of the MEAD system 500F, a waveguide 424 is disposed proximate the upper side of the MEAD system 500F, and/or a waveguide 424 is disposed proximate the lower side of the MEAD system 500F.

The MEAD system 500F may be disposed within a housing or within ducting. The MEAD system 500F may include a first waveguide 424 proximate a left side of the MEAD system 500F and a second waveguide 424 proximate a right side of the MEAD system 500F. The waveguides 424 may be at least partially disposed within the housing or ducting of the MEAD system 500F or may be disposed outside of the housing or ducting of the MEAD system 500F. Each waveguide 424 may be coupled to a microwave generator 420 via a magnetron tube 426. In some embodiments, each waveguide 424 has a separate microwave generator 420 and magnetron tube 426. In some embodiments, the waveguides 424 may have a common microwave generator 420.

FIG. 5G is a cross-sectional view of a portion of a ventilation system 501 including a MEAD system 500G (e.g. MEAD system 500F), according to certain embodiments. The ventilation system 501 may include one or more segments of ducting 504. The MEAD system 500G may be coupled to the ducting 504 (e.g., airflow through ducting 504 goes through MEAD system 500G) or may be disposed in the ducting. The MEAD system includes a multi-component filter 130 and one or more waveguides 424. A magnetron tube 426 and microwave generator 420 are coupled to each waveguide 424 to provide microwave energy through the waveguide 424 to the multi-component filter 130.

In some embodiments, the waveguide 424 is in the middle of the multi-component filter 130 (e.g., see FIGS. 3A-B). In some embodiments, the waveguide 424 is to the side of the multi-component filter 130 (e.g., see FIGS. 4A-B, FIGS. 5F-G). In some embodiments, the waveguide 424 is to the side of the multi-component filter 130 and the multi-component filter 130 is angled (e.g., see FIG. 5G) to not be perpendicular to airflow. By angling the multi-component filter 130, filtration area of the multi-component filter 130 may be increased which may increase life of the multi-component filter 130. The one or more waveguides 424 may be disposed proximate a sidewall of the ducting and/or housing of MEAD 424 to reduce the obstruction of the airflow.

FIG. 5H is a cross-sectional view of a MEAD system 500F. MEAD system 500F may be similar to MEAD systems 500F-G of FIGS. 5F-G. MEAD system 500H may include a multi-component filter 130 and a waveguide 424 to provide microwave energy to the multi-component filter 130. In some embodiments, waveguide 424 is disposed proximate a center axis of the housing 410 and/or ducting 504. The waveguide 424 may be coupled to a microwave generator 420 via a magnetron tube 426.

MEAD system 500H may include an interface 590. The interface 590 may be used to control, schedule, receive information, provide information, etc. for the MEAD system 500H. A housing may be coupled to the interface 590 and other components (e.g., microwave generator, microwave magnetron, capacitor, cooling fan, etc.).

The MEAD system 500H may include an active energy distributor 580A and/or a passive energy distributor 580B. The active energy distributor 580A and/or passive energy distributor 580B may adjust (e.g., reflect, move, break up, randomize, cause to bounce in different directions) microwaves provided via waveguide 424 (e.g., break up microwave field). This reduces or prevents cancelation of microwaves provided via waveguide 424.

Active energy distributor 580A may be a mechanical device, such as a stirrer (e.g., motor moving a propeller blade). The active energy distributor 580A may have one or more blades configured to be actuated (e.g., rotated) to reflect (e.g., move, cause to bounce, break up) microwaves provided via waveguide 424.

Passive energy distributor 580B may include one or more features (e.g., protrusions, recesses, reflectors, bumps, fins, impressions, dimples, etc.) disposed on the inside walls of the housing 410 and/or ducting 504 of MEAD system 500H. The features may be non-uniformly distributed. The features may be disposed between the screen 582A and the multi-component filter 130. The features may be disposed between screen 582A and screen 582B.

FIG. 5I is a cross-sectional view of a portion of a ventilation system 501 including a MEAD system 500I (e.g., MEAD system 500H), according to certain embodiments. The ventilation system 500I may include one or more segments of ducting 504. The MEAD system 500I may be coupled to the ducting 504 (e.g., airflow through ducting 504 goes through MEAD system 500I) or may be disposed in the ducting 504. The MEAD system includes a multi-component filter 130 and a waveguide 424. A magnetron tube 426 and microwave generator 420 are coupled the waveguide 424 to provide microwave energy through the waveguide 424 to the multi-component filter 130.

In some embodiments, the waveguide 424 is in the middle of the multi-component filter 130 (e.g., see FIGS. 3A-B). The MEAD system 500I may include an active energy distributor 580A and/or a passive energy distributor 580B to adjust (e.g., reflect, move, break up, randomize, cause to bounce in different directions) microwaves provided via waveguide 424 (e.g., break up microwave field).

In some embodiments, the waveguide 424 and the multi-component filter 130 are disposed between screen 582A and screen 582B. Screens 582A-B may be metal (e.g., copper, aluminum, steel, etc.) microwave containment grids. Screen 582A and screen 582B may prevent the microwave energy from leaving the MEAD system 500I. Screens 582A-B may form holes to allow airflow 542 through the screens 582A-B. The holes may be circular, triangular, rectangular, hexagon-shaped, etc. The holes (e.g., hexagon-shaped holes) may have a maximum height of one half or one third the wavelength of the microwave energy (e.g., lambda over 2, lambda over 3). In some embodiments, for a microwave wavelength of 2.4 MHz, the holes would have a maximum height of one fourth inch or one eight inch.

Screen 582A may be disposed proximate (e.g., directly contacting) waveguide 424. Screen 582A and/or screen 582B may be substantially vertical, curved, angled, etc. Screen 582A may be substantially vertical and screen 582B may be curved (e.g., form a half-circle) so that the top and bottom edge of the screen 582B are closer to screen 582A and the middle of screen 582B is further away from screen 582A.

Airflow may be through ducting 504, then through screen 582B, then through multi-component filter 130, then past waveguide 424, then through screen 582A, and then through the ducting 504. Having the multi-component filter 130 before waveguide 424 and screen 582A may prevent particle (e.g., fiber, contaminant) build up on waveguide 424 and screen 582A. Screen 582B may collect particles (e.g., fibers, contaminants) and may be replaced and/or cleaned periodically. In some embodiments, the screen 582 is coupled to multi-component filter 130.

FIG. 6 is a block diagram illustrating a computer system 600, according to certain embodiments. In some embodiments, the computer system 600 is a controller of the MEAD system (controller 150 of MEAD system 100). In some embodiments, the processor 602 is the controller of the MEAD system (controller 150 of MEAD system 100).

In some embodiments, computer system 600 is connected (e.g., via a network, such as a Local Area Network (LAN), an intranet, an extranet, or the Internet) to other computer systems. In some embodiments, computer system 600 operates in the capacity of a server or a client computer in a client-server environment, or as a peer computer in a peer-to-peer or distributed network environment. In some embodiments, computer system 600 is provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 600 includes a processing device 602, a volatile memory 604 (e.g., Random Access Memory (RAM)), a non-volatile memory 606 (e.g., Read-Only Memory (ROM) or Electrically-Erasable Programmable ROM (EEPROM)), and a data storage device 616, which communicate with each other via a bus 608.

In some embodiments, processing device 602 is provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor).

In some embodiments, computer system 600 further includes a network interface device 622 (e.g., coupled to network 674). In some embodiments, computer system 600 also includes a video display unit 610 (e.g., an LCD), an alphanumeric input device 612 (e.g., a keyboard), a cursor control device 614 (e.g., a mouse), and a signal generation device 620.

In some implementations, data storage device 616 includes a non-transitory computer-readable storage medium 624 on which store instructions 626 encoding any one or more of the methods or functions described herein, including instructions for implementing methods described herein.

In some embodiments, instructions 626 also reside, completely or partially, within volatile memory 604 and/or within processing device 602 during execution thereof by computer system 600, hence, in some embodiments, volatile memory 604 and processing device 602 also constitute machine-readable storage media.

While computer-readable storage medium 624 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

In some embodiments, the methods, components, and features described herein are implemented by discrete hardware components or are integrated in the functionality of other hardware components such as ASICS, FPGAs, DSPs or similar devices. In some embodiments, the methods, components, and features are implemented by firmware modules or functional circuitry within hardware devices. In some embodiments, the methods, components, and features are implemented in any combination of hardware devices and computer program components, or in computer programs.

Unless specifically stated otherwise, terms such as “generating,” “providing,” “causing,” “removing,” “determining,” “transmitting,” “receiving,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. In some embodiments, the terms “first,” “second,” “third,” “fourth,” etc. as used herein are meant as labels to distinguish among different elements and do not have an ordinal meaning according to their numerical designation.

Examples described herein also relate to an apparatus for performing the methods described herein. In some embodiments, this apparatus is specially constructed for performing the methods described herein, or includes a general purpose computer system selectively programmed by a computer program stored in the computer system. Such a computer program is stored in a computer-readable tangible storage medium.

Some of the methods and illustrative examples described herein are not inherently related to any particular computer or other apparatus. In some embodiments, various general purpose systems are used in accordance with the teachings described herein. In some embodiments, a more specialized apparatus is constructed to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ±10%.

Although the operations of the methods herein are shown and described in a particular order, the order of operations of each method may be altered so that certain operations may be performed in an inverse order so that certain operations may be performed, at least in part, concurrently with other operations. In another embodiment, instructions or sub-operations of distinct operations may be in an intermittent and/or alternating manner.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A microwave enhanced air disinfection (MEAD) device comprising:
a housing;
a microwave generator coupled to the housing, wherein the microwave generator is configured to generate microwave energy;
a multi-component filter disposed in the housing, wherein the multi-component filter is configured to collect contaminants from airflow, and wherein at least a portion of the contaminants from the airflow is to be destroyed at least one of directly or indirectly via the microwave energy.

2. The MEAD device of claim 1 further comprising:
a waveguide at least partially disposed in the housing, wherein the waveguide is configured to receive the microwave energy from the microwave generator, wherein the waveguide is configured to direct the microwave energy toward the multi-component filter.

3. The MEAD device of claim 2 further comprising:
a magnetron tube coupled to the microwave generator, wherein the magnetron tube is at least partially disposed within the waveguide, and wherein the magnetron tube is configured to direct the microwave energy from the microwave generator into the waveguide.

4. The MEAD device of claim 1, wherein the multi-component filter comprises:
a microwave-absorbing layer configured to collect a first subset of the contaminants from the airflow, wherein the microwave absorbing layer is configured to be activated by the microwave energy to destroy the first subset of the contaminants from the airflow; and
a high-efficiency particulate air (HEPA) filter configured to collect a second subset of the contaminants from the airflow.

5. The MEAD device of claim 4, wherein the multi-component filter comprises:
a molecular sieve disposed between the microwave-absorbing layer and the HEPA filter to collect at least a third subset of the contaminants from the airflow.

6. The MEAD device of claim 5, wherein the microwave-absorbing layer comprises a metal oxide or silicon carbide (SiC), and wherein the molecular sieve comprises zeolites.

7. The MEAD device of claim 1, wherein the multi-component filter comprises a heterogeneous mix of two or more filter materials, wherein each of the two or more filter materials perform a different function.

8. The MEAD device of claim 1, wherein the multi-component filter has a thickness of four inches or less.

9. The MEAD device of claim 1, wherein:
the multi-component filter comprises a desiccant material configured to absorb moisture comprising the at least a portion of the contaminants; and
the microwave energy regenerates the desiccant material by causing the moisture to become steam to exit the MEAD device.

10. The MEAD device of claim 9, wherein the microwave energy causes the moisture to become the steam and destroys the at least a portion of the contaminants without directly heating the desiccant material.

11. The MEAD system of claim 9 further comprising:
one or more sensors configured to provide sensor data; and
a controller configured to:
determine, based on the sensor data, that the desiccant material is to be regenerated; and
cause the microwave generator to generate the microwave energy to regenerate the desiccant material.

12. The MEAD system of claim 1, wherein the multi-component filter comprises:
a first silicon carbide (SiC) layer configured to absorb the microwave energy to destroy at least a first portion of the contaminants;
a zeolites and metal oxides layer configured to catalyze a reaction to destroy at least a second portion of the contaminants;
a desiccant material layer configured to absorb moisture comprising at least a third portion of the contaminants, wherein the at least a third portion of the contaminants is to be destroyed responsive to the microwave energy causing the moisture to become steam; and
a second SiC layer configured to absorb the microwave energy to destroy at least a fourth portion of the contaminants.

13. The MEAD system of claim 13, wherein the zeolites and metal oxides layer and the desiccant material layer are disposed between the first SiC layer and the second SiC layer.

14. The MEAD system of claim 1 further comprising at least one of an active energy distributor or a passive energy distributor configured to reflect the microwave energy within the MEAD system.

15. The MEAD system of claim 2 further comprising a first screen and a second screen, wherein the multi-component filter and the waveguide are disposed between the first screen and the second screen, wherein the airflow is to flow through the first screen and the second screen, and wherein the first screen and the second screen are to prevent the microwave energy from leaving the MEAD system.

16. The MEAD system of claim 15, wherein the second screen and the first screen form openings that are about 0.125 to 0.25 inches in height.

17. The MEAD system of claim 15, wherein the second screen and the first screen form openings that are hexagon-shaped.

18. A microwave enhanced air disinfection (MEAD) system comprising:
a housing configured to receive airflow;
a microwave generator coupled to the housing, wherein the microwave generator is configured to intermittently generate microwave energy; and
a filter disposed in the housing, wherein the filter is configured to collect contaminants from the airflow, and wherein at least a first portion of the contaminants from the airflow is to be destroyed at least one of directly or indirectly via the microwave energy.

19. The MEAD system of claim 18, wherein the filter comprises a microwave-absorbing layer coupled to a backing layer, wherein the microwave-absorbing layer is configured to collect the first portion of the contaminants to be destroyed, and wherein the backing layer is configured to collect a second portion of the contaminants, wherein the backing layer is configured to be heated to about 80 to about 150 degrees Celsius via the microwave energy.

20. The MEAD system of claim 19, wherein:
the microwave-absorbing layer comprises an inlet microwave screen coated with microwave-absorbing material;
the backing layer is disposed between the microwave-absorbing layer and an outlet microwave screen; and
the inlet microwave screen and the outlet microwave screen are configured to prevent leaking of microwave energy.

21. The MEAD system of claim 18 further comprising:
a waveguide at least partially disposed in the housing, wherein the waveguide is configured to receive the microwave energy from the microwave generator, and wherein the waveguide is configured to direct the microwave energy toward the filter; and
a magnetron tube coupled to the microwave generator, wherein the magnetron tube is at least partially disposed within the waveguide, and wherein the magnetron tube is configured to direct the microwave energy from the microwave generator into the waveguide.

22. The MEAD system of claim 18, wherein the MEAD system is disposed within a ventilation system and the MEAD system provides less than about 0.5 inches of water gauge of pressure drop in the ventilation system.

23. A microwave enhanced air disinfection (MEAD) device comprising:
a housing;
a microwave generator coupled to the housing, wherein the microwave generator is configured to generate microwave energy;
a cylindrical slotted waveguide disposed in the housing, wherein the cylindrical slotted waveguide is configured to direct the microwave energy; and
a multi-component filter disposed around the cylindrical slotted waveguide, wherein the multi-component filter is disposed in the housing, wherein the multi-component filter is configured to collect contaminants from airflow, and wherein at least a portion of the contaminants from the airflow is to be destroyed at least one of directly or indirectly via the microwave energy.

24. The MEAD device of claim 23 further comprising
a magnetron tube coupled to the microwave generator, wherein the magnetron tube is at least partially disposed within the cylindrical slotted waveguide, and wherein the magnetron tube is configured to direct the microwave energy from the microwave generator into the cylindrical slotted waveguide, wherein the multi-component filter comprises:
a microwave-absorbing layer configured to collect a first subset of the contaminants from the airflow, wherein the microwave absorbing layer is configured to be activated by the microwave energy to destroy the first subset of the contaminants from the airflow, wherein the microwave-absorbing layer is configured to destroy the first subset of the contaminants by oxidizing the first subset of the contaminants; and
a high-efficiency particulate air (HEPA) filter configured to collect a second subset of the contaminants from the airflow.
